SEQUENCE LISTING (56 sequences)

SEQ ID NO: 1 - DNA, 171 bp, Schizosaccharomyces pombe
  CDS (1)..(171)
atgaagatcaccgctgtcattgcccttttattctcacttgctgctgcctcacctattccagttgccgatcctggtgtggtttcagttagcaagtcatatgctgatttccttcgtgtttaccaaagttggaacacttttgctaatcctgatagacccaacttgaaaaagcgc

SEQ ID NO: 2 - protein, 57 aa, Schizosaccharomyces pombe
MKITAVIALLFSLAAASPIPVADPGVVSVSKSYADFLRVYQSWNTFANPDRPNLKKR

SEQ ID NO: 3 - DNA, 60 bp, Schizosaccharomyces pombe
  CDS (1)..(60)
  sig_peptide (1)..(60)
atgaagatcaccgctgtcattgcccttttattctcacttgctgctgcctcacctattcca

SEQ ID NO: 4 - protein, 20 aa, Schizosaccharomyces pombe
MKITAVIALLFSLAAASPIP

SEQ ID NO: 5 - DNA, 81 bp, Schizosaccharomyces pombe
  CDS (1)..(81)
aagtcatatgctgatttccttcgtgtttaccaaagttggaacacttttgctaatcctgatagacccaacttgaaaaagcgc

SEQ ID NO: 6 - protein, 27 aa, Schizosaccharomyces pombe
KSYADFLRVYQSWNTFANPDRPNLKKR

SEQ ID NO: 7 - DNA, 78 bp, Schizosaccharomyces pombe
  CDS (1)..(78)
  sig_peptide (1)..(60)
atgaagatcaccgctgtcattgcccttttattctcacttgctgctgcctcacctattccagttgccgatcctggtgtg

SEQ ID NO: 8 - protein, 26 aa, Schizosaccharomyces pombe
MKITAVIALLFSLAAASPIPVADPGV

SEQ ID NO: 9 - DNA, 606 bp, Schizosaccharomyces pombe
  CDS (1)..(606)
atgaagatcaccgctgtcattgcccttttattctcacttgctgctgcctcacctattccagttgccgatcctggtgtggtttcagttagcaagtcatatgctgatttccttcgtgtttaccaaagttggaacacttttgctaatcctgatagacccaacttgaaaaagcgcgaattcgaagctgctcccgcaaaaacttatgctgatttccttcgtgcttatcaaagttggaacacttttgttaatcctgacagacccaatttgaaaaagcgtgagtttgaagctgccccagagaagagttatgctgatttccttcgtgcttaccatagttggaacacttttgttaatcctgacagacccaacttgaaaaagcgcgaattcgaagctgctcccgcaaaaacttatgctgatttccttcgtgcttaccaaagttggaacacttttgttaatcctgacagacccaacttgaaaaagcgcactgaagaagatgaagagaatgaggaagaggatgaagaatactatcgctttcttcagttttatatcatgactgtcccagagaattccactattacagatgtcaatattactgccaaatttgagagctaa

SEQ ID NO: 10 - protein, 201 aa, Schizosaccharomyces pombe
MKITAVIALLFSLAAASPIPVADPGVVSVSKSYADFLRVYQSWNTFANPDRPNLKKREFEAAPAKTYADFLRAYQSWNTFVNPDRPNLKKREFEAAPEKSYADFLRAYHSWNTFVNPDRPNLKKREFEAAPAKTYADFLRAYQSWNTFVNPDRPNLKKRTEEDEENEEEDEEYYRFLQFYIMTVPENSTITDVNITAKFES

SEQ ID NO: 11 - DNA, 156 bp, Unknown
  HA-tag
  CDS (1)..(156)
ctggttccgcgtggatccatcgaaggtcgtggcggccgcatcttttacccatacgatgttcctgactatgcgggctatccctatgacgtcccggactatgcaggatcctatccatatgacgttccagattacgctgctcagtgcggccgctaatag

SEQ ID NO: 12 - protein, 50 aa, Unknown
  HA-tag
LVPRGSIEGRGGRIFYPYDVPDYAGYPYDVPDYAGSYPYDVPDYAAQCGR

SEQ ID NO: 13 - DNA, 354 bp, Aspergillus nidulans
  CDS (1)..(354)
ctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgccggtaccaaggctgagtaa

SEQ ID NO: 14 - protein, 117 aa, Aspergillus nidulans
LPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 15 - DNA, 408 bp, Aspergillus nidulans
  CDS (1)..(408)
atgcgcttcatcgtctctctcctcgccttcactgccgcggccaccgcaaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgccggtaccaaggctgagtaa

SEQ ID NO: 16 - protein, 135 aa, Aspergillus nidulans
MRFIVSLLAFTAAATATALPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAE

SEQ ID NO: 17 - DNA, 678 bp, Artificial Sequence
  Fusion protein
  CDS (1)..(678)
atgaagatcaccgctgtcattgcccttttattctcacttgctgctgcctcacctattccagttgccgatcctggtgtggtttcagttagcaagtcatatgctgatttccttcgtgtttaccaaagttggaacacttttgctaatcctgatagacccaacttgaaaaagcgcctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcgctctcgtcgaccacactgaggaaggccccgtctgcaagaacatcgtcgcttgctgccctgagggaaccaccaactgtgttgccgtcgacaacgctggcgccggtaccaaggctgagctggttccgcgtggatccatcgaaggtcgtggcggccgcatcttttacccatacgatgttcctgactatgcgggctatccctatgacgtcccggactatgcaggatcctatccatatgacgttccagattacgctgctcagtgcggccgctaatag

SEQ ID NO: 18 - protein, 224 aa, Artificial Sequence
  Fusion protein
MKITAVIALLFSLAAASPIPVADPGVVSVSKSYADFLRVYQSWNTFANPDRPNLKKRLPASAAKNAKLATSAAFAKQAEGTTCNVGSIACCNSPAETNNDSLLSGLLGAGLLNGLSGNTGSACAKASLIDQLGLLALVDHTEEGPVCKNIVACCPEGTTNCVAVDNAGAGTKAELVPRGSIEGRGGRIFYPYDVPDYAGYPYDVPDYAGSYPYDVPDYAAQCGR

SEQ ID NO: 19 - protein, 131 aa, Streptomyces coelicolor
MLKKAMVAAAAAASVIGMSAAAAPQALAIGDDNGPAVANGNGAESAFGNSATKGDMSPQLSLVEGTLNKPCLGVEDVNVAVINLVPIQDINVLADDLNQQCADNSTQAKRDGALSHVLEDLSVLSANGEGR

SEQ ID NO: 20 - protein, 133 aa, Streptomyces coelicolor
MIKKVVAYAAIAASVMGASAAAAPQAMAIGDDSGPVSANGNGASQYFGNSMTTGNMSPQMALIQGSFNKPCIAVSDIPVSVIGLVPIQDLNVLGDDMNQQCAENSTQAKRDGALAHLLEDVSILSSNGEGGKG

SEQ ID NO: 21 - protein, 112 aa, Agaricus bisporus
MISRVLVAALVALPALVTATPAPGKPKASSQCDVGEIHCCDTQQTPDHTSAAASGLLGVPINLGAFLGFDCTPISVLGVGGNNCAAQPVCCTGNQFTALINALDCSPVNVNL

SEQ ID NO: 22 - protein, 119 aa, Agaricus bisporus
MVSTFITVAKTLLVALLFVNINIVVGTATTGKHCSTGPIECCKQVMDSKSPQATELLTKNGLGLGVLAGVKGLVGANCSPITAIGIGSGSQCSGQTVCCQNNNFNGVVAIGCTPINANV

SEQ ID NO: 23 - DNA, 32 bp, Artificial Sequence
  PCR primer
cagctgggtctgctcgctctcgtcgaccacac

SEQ ID NO: 24 - DNA, 32 bp, Artificial Sequence
  PCR primer
gtgtggtcgacgagagcgagcagacccagctg

SEQ ID NO: 25 - DNA, 30 bp, Artificial Sequence
  PCR primer
gagggaaccaccaactgtgttgccgtcgac

SEQ ID NO: 26 - DNA, 30 bp, Artificial Sequence
  PCR primer
gtcgacggcaacacagttggtggttccctc

SEQ ID NO: 27 - DNA, 34 bp, Artificial Sequence
  PCR primer
taataactcgagatgcgcttcatcgtctctctcc

SEQ ID NO: 28 - DNA, 33 bp, Artificial Sequence
  PCR primer
taataaggatccttactcagccttggtaccggc

SEQ ID NO: 29 - DNA, 30 bp, Artificial Sequence
  PCR primer
ggtaccaaggctgagctggttccgcgtgga

SEQ ID NO: 30 - DNA, 30 bp, Artificial Sequence
  PCR primer
tccacgcggaaccagctcagccttggtacc

SEQ ID NO: 31 - DNA, 36 bp, Artificial Sequence
  PCR primer
attattccatggctattagcggccgcactgagcagc

SEQ ID NO: 32 - DNA, 30 bp, Artificial Sequence
  PCR primer
gcctcacctattccactcccggcctctgcc

SEQ ID NO: 33 - DNA, 30 bp, Artificial Sequence
  PCR primer
ggcagaggccgggagtggaataggtgaggc

SEQ ID NO: 34 - DNA, 49 bp, Artificial Sequence
  PCR primer
taatttctcgagatgaagatcaccgctgtcattgcccttttattctcac

SEQ ID NO: 35 - DNA, 33 bp, Artificial Sequence
  PCR primer
gttgccgatcctggtgtgctcccggcctctgcc

SEQ ID NO: 36 - DNA, 33 bp, Artificial Sequence
  PCR primer
cacaccaggatcggcaactggaataggtgaggc

SEQ ID NO: 37 - DNA, 30 bp, Artificial Sequence
  PCR primer
aacttgaaaaagcgcctcccggcctctgcc

SEQ ID NO: 38 - DNA, 35 bp, Artificial Sequence
  PCR primer
ggcagaggccgggaggcgctttttcaagttgggtc

SEQ ID NO: 39 - DNA, 552 bp, Aspergillus nidulans
  CDS (1)..(288)
  CDS (508)..(549)
  intron (456)..(507)
  CDS (381)..(455)
  Intron (289)..(380)
atgcgcttcatcgtctctctcctcgccttcactgccgcggccaccgcaaccgccctcccggcctctgccgcaaagaacgcgaagctggccacctcggcggccttcgccaagcaggctgaaggcaccacctgcaatgtcggctcgatcgcttgctgcaactcccccgctgagaccaacaacgacagtctgttgagcggtctgctcggtgctggccttctcaacgggctctcgggcaacactggcagcgcctgcgccaaggcgagcttgattgaccagctgggtctgctcggtacgtgatccccactcagtcgctcccggagaggctgagggaagacgagcgacggtctagaaatggtgtgctaatagatgcatgtgtgcagctctcgtcgaccacactgaggaaggccccgtctgcaagaacatcgtcgcttgctgccctgagggaaccaccaacgtacgtctttcagatctgctacaagtgaggcgatcaaaactaacatattccagtgtgttgccgtcgacaacgctggcgccggtaccaaggctgagtaa

SEQ ID NO: 40 - protein, 135 aa, Aspergillus nidulans
MRFIVSLLAFTAAATATALPASAAKNAKLATSAAFAKQAEGTTCNVGSIACCNSPAETNNDSLLSGLLGAGLLNGLSGNTGSACAKASLIDQLGLLLSSTTLRKAPSARTSSLAALREPPTCVAVDNAGAGTKAE

SEQ ID NO: 41 - DNA, 34 bp, Artificial Sequence
  PCR primer
taataaggatccatgcgcttcatcgtctctctcc

SEQ ID NO: 42 - DNA, 129 bp, Schizosaccharomyces pombe
  CDS (1)..(126)
atggactcaatggctaactccgtttcttcctcctctgtcgtcaacgctggcaacaagcctgctgaaactcttaacaagaccgttaagaattatacccccaaggttccttacatgtgtgtcattgcataa

SEQ ID NO: 43 - protein, 42 aa, Schizosaccharomyces pombe
MDSMANSVSSSSVVNAGNKPAETLNKTVKNYTPKVPYMCVIA

SEQ ID NO: 44 - DNA, 27 bp, Schizosaccharomyces pombe
tatacccccaaggttccttacatgtgt

SEQ ID NO: 45 - DNA, 135 bp, Schizosaccharomyces pombe
  CDS (1)..(132)
atggactccattgcaactaacactcattcttcatccattgtcaatgcctacaacaacaatcctaccgatgttgtaaaaactcaaaacattaaaaattatactccaaaggttccttatatgtgtgtaattgcttaa

SEQ ID NO: 46 - protein, 44 aa, Schizosaccharomyces pombe
MDSIATNTHSSSIVNAYNNNPTDVVKTQNIKNYTPKVPYMCVIA

SEQ ID NO: 47 - DNA, 27 bp, Schizosaccharomyces pombe
tatactccaaaggttccttatatgtgt

SEQ ID NO: 48 - DNA, 126 bp, Schizosaccharomyces pombe
  CDS (1)..(123)
atggactcaatggctaacactgtttcttcctccgtcgttaacactggcaacaagccttctgaaactcttaacaagactgttaagaattatacccccaaggttccttacatgtgtgtcattgcataa

SEQ ID NO: 49 - protein, 41 aa, Schizosaccharomyces pombe
MDSMANTVSSSVVNTGNKPSETLNKTVKNYTPKVPYMCVIA

SEQ ID NO: 50 - DNA, 27 bp, Schizosaccharomyces pombe
tatacccccaaggttccttacatgtgt

SEQ ID NO: 51 - protein, 9 aa, Schizosaccharomyces pombe
YTPKVPYMC

SEQ ID NO: 52 - DNA, 586 bp, Aspergillus nidulans
  Intron (471)..(530)
  Intron (338)..(389)
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctggtcagttcttcgaaaatcactttcgtgatgccccaatgctaacaattaccagtcctcattggcatccaagatcttgtcaaccagaagtgcaagcaaaacattgcctgctgccagaactccccctccagcgcggtatgttcccttgttttacagcttattcacttaaaccgattaatctaacaacgctcacaggatggcaaccttattggtgtcggtctcccttgcgttgcccttggctccatcctctaa

SEQ ID NO: 53 - DNA, 474 bp, Aspergillus nidulans
  CDS (1)..(471)
atgaagttctccattgctgccgctgtcgttgctttcgccgcctccgtcgcggccctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctctaa

SEQ ID NO: 54 - protein, 157 aa, Aspergillus nidulans
MKFSIAAAVVAFAASVAALPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

SEQ ID NO: 55 - DNA, 420 bp, Aspergillus nidulans
  CDS (1)..(417)
ctccctcctgcccatgattcccagttcgctggcaatggtgttggcaacaagggcaacagcaacgtcaagttccctgtccccgaaaacgtgaccgtcaagcaggcctccgacaagtgcggtgaccaggcccagctctcttgctgcaacaaggccacgtacgccggtgacaccacaaccgttgatgagggtcttctgtctggtgccctcagcggcctcatcggcgccgggtctggtgccgaaggtcttggtctcttcgatcagtgctccaagcttgatgttgctgtcctcattggcatccaagatcttgtcaaccagaagtgcaagcaaaacattgcctgctgccagaactccccctccagcgcggatggcaaccttattggtgtcggtctcccttgcgttgcccttggctccatcctctaa

SEQ ID NO: 56 - protein, 139 aa, Aspergillus nidulans
LPPAHDSQFAGNGVGNKGNSNVKFPVPENVTVKQASDKCGDQAQLSCCNKATYAGDTTTVDEGLLSGALSGLIGAGSGAEGLGLFDQCSKLDVAVLIGIQDLVNQKCKQNIACCQNSPSSADGNLIGVGLPCVALGSIL

(19) United States
(12) Patent Application Publication
(10) Pub. No.: US 2007/0077619 A1
(43) Pub. Date: Apr. 5, 2007

(54) Secretion of proteins from yeasts

(75) Inventors: Kai Ostermann, Dresden (DE); Gerhard Rodel, Karlsfeld (DE)

Correspondence Address: CONNOLLY BOVE LODGE & HUTZ, LLP, P O BOX 2207, WILMINGTON, DE, 19899, US

(73) Assignee: BASF Aktiengesellscaft, Ludwigshafen (DE)

(21) Appl. No.: 10/572,189

(22) PCT Filed: Sep. 15, 2004
(86) PCT No.: PCT/EP04/10348

§ 371 Date: Mar. 15, 2006

(30) Foreign Application Priority Data
Sep. 15, 2003 (DE) 10342794.5

(51) Int. Cl. C11D 3/386; C07H 21/04; C12P 21/06; C12N 15/74; C07K 14/39

(52) U.S. Cl. 435/69.1; 435/254.2; 435/483; 530/359; 536/23.7; 510/320

(57) ABSTRACT

The present invention relates to expression constructs encompassing the nucleic acid sequence coding for a shuttle peptide construct processable by yeast cells; to corresponding expression vectors comprising such constructs; to methods carried out with the aid thereof for recombinant preparation of target proteins; to hosts transformed therewith; to shuttle peptides and nucleic acid sequences coding therefor; to nucleic acid sequences coding for such shuttle peptides fused to a foreign protein; to hydrophobin proteins prepared using shuttle peptides of this kind and to the use of hydrophobins for the coating of objects such as, for example, leather.

The present invention relates to expression constructs encompassing the nucleic acid sequence coding for a shuttle peptide construct processable by yeast cells; to corresponding expression vectors comprising such constructs; to methods carried out with the aid thereof for recombinant preparation of target proteins; to hosts transformed therewith; to shuttle peptides and nucleic acid sequences coding therefor; to nucleic acid sequences coding for such shuttle peptides fused to a foreign protein; to hydrophobin proteins prepared using shuttle peptides of this kind and to the use of hydrophobins for the coating of objects such as, for example, leather.

### PRIOR ART

a) Expression in Yeasts

Yeasts are widely used as hosts for heterologous protein expression. The reason for this is that a yeast expression system has several advantages, since yeasts can grow at a higher density compared to bacteria and other eukaryotic cells and are capable of protein glycosylation and posttranslational modification. Moreover, the products produced and secreted by yeasts can be purified in a simple manner, because yeasts are highly resistant to cell lysis and the growth medium usually contains low amounts of foreign protein. In addition, yeasts can grow faster than other eukaryotic cells at high density on inexpensive nutrient media.

Numerous different approaches for expressing and secreting heterologous proteins in yeasts can be found in the prior art. Thus, for example, U.S. Pat. No. 5,642,487 describes a method for recombinant production of proteins in yeasts, which comprises transforming yeast with an expression cassette coding for a structural element which encodes a leader sequence of an animal peptide neurohormone, an adaptor sequence producing an α-helical structure, a processing signal and a structural gene.

It is furthermore known from the prior art to use regulatory elements of the gene of the α-factor, a pheromone produced by yeasts, for controlling expression of heterologous proteins in yeasts. Thus, for example, α-factor signal leader peptide sequences were used for expressing heterologous proteins (cf. e.g. U.S. Pat. No. 5,010,182).

Moreover, the published US patent application US 2003/0077831 discloses an expression vector for expressing heterologous proteins in yeasts which encompasses, flanked by suitable transcription start and translation start and termination sequences, the coding sequence for a hybrid precursor polypeptide whose elements comprise the signal peptide and the leader peptide of a protein secreted by yeasts and also a heterologous protein flanked by N-terminal and C-terminal propeptide sequences of said heterologous protein.

b) Hydrophobins

Hydrophobins are small cystein-rich proteins comprising approximately 100 amino acid residues and having interesting technical propterties. They are capable of rendering hydrophobic surfaces hydrophilic. They make hydrophilic surfaces become hydrophobic.

However, there are a number of patents on hydrophobins and application thereof: thus, for example, WO-A-96/41882 describes hydrophobins from edible fungi (cf. SEQ ID NO:21 and 22). WO-A-00/58342 relates to purification of hydrophobin-containing fusion proteins by phase extraction. WO-A-01/57066 describes stabilization, solubilization and, related thereto, improved application of hydrophobins due to sulfite treatment. WO-A-01/57076 describes purification of hydrophobin via adsorption to Teflon beads and elution by means of a detergent such as Tween at low temperatures. WO-A-01/57528 describes fixing hydrophobins to surfaces by applying Tween and temperatures up to 85 degrees Celsius.

WO-A-01/74864 describes untypical hydrophobins (only one disulfide bridge), referred to as RdlA and RdlB (cf. SEQ ID NO:19 and 20), of filamentous bacteria, in particular Streptomyces sp. The hydrophobin is used for surface treatment of various objects such as windows, contact lenses, vehicle bodies. It is furthermore suggested to produce the proteins described there in a recombinant host which releases said proteins into the medium. After removing the host, the hydrophobin-containing medium is supposedly suitable for surface coating. Experimental evidence of actual expression and secretion is not provided.

### SUMMARY OF THE INVENTION

It is an object of the present invention to provide means which make it possible to secrete homologous or, in particular, heterologous proteins expressed in yeast, in particular Schizosaccharomyces pombe, from the yeast cells into the surrounding medium. In particular, means should be provided which enable recombinantly produced hydrophobin to be secreted from the host cell.

We have found that this object is achieved by providing an expression construct which comprises the nucleic acid sequence coding for a shuttle peptide construct which is processable by yeast cells, has the formula
(Sig-SP),
and comprises in 5′-3′ orientation the nucleic acid sequences coding for
a) a signal peptide (Sig) processably linked to
b) at least one shuttle peptide (SP) secretable by said yeast cells.

Achieving the above object is illustrated in a model way by the example of the Aspergillus nidulans hydrophobin DewA (mature protein according to SEQ ID NO:14 with coding sequence according to SEQ ID NO:13; pre-protein with signal sequence: SEQ ID NO:16 with coding nucleic acid sequence according to SEQ ID NO:15) as heterologous target protein (Targ). This protein is a representative of class I hydrophobins, i.e. of secreted fungal coat proteins capable of self-assembling.

In particular, the DNA sequence (SEQ ID NO: 13) coding for the target protein (DewA) is fused to the 3′-terminal end of the DNA sequence (SEQ ID NO:5 for mature P factor) coding for an S. pombe peptide pheromone (P factor; amino acid sequence according to SEQ ID NO:6 for mature P factor). The resulting fusion protein comprises all signal sequences required for secreting said pheromone and the target protein fused thereto, in particular the removable signal peptide (SEQ ID NO:4). Secretion involves proteolytic processing of the fusion protein. As a consequence, the pheromone (P factor) (SEQ ID NO:6) and the target protein (hydrophobin; SEQ ID NO: 14) are secreted separately into the medium.

The finding of the invention is surprising insofar as the actual regulatory elements of the P-factor pre-protein (N-terminal of the mature pheromone) are evidently not sufficient to control secretion of the target protein by the yeast cells. Only the use of a construct in which an additional, co-secreting protein component (the mature pheromone) is processably located upstream of the target protein to be secreted enables said target protein to be secreted into the culture medium in the desired manner.

### DETAILED DESCRIPTION OF THE INVENTION

a) General Information

The protein sequences are usually indicated in the “one-letter code” in the description and the figures.

A protein which is intracellularly expressed by a host cell, in particular by yeasts, and which is secreted from the cell via endogenous cell mechanisms through the cell membrane, preferably into the surrounding medium is “secretable” in accordance with the present invention.

A protein precursor (i.e. a protein in its originally expressed form, such as, for example a pre-protein, with N- and/or C-terminal peptide sequences no longer present in the mature processed protein) is “processable” in accordance with the present invention, if it can be converted to the mature form by proteolytic processes inside and/or outside the host cell.

A “processable linkage” is present if individual protein sections in a protein to be processed are linked via peptide bonds which can be cleaved by a proteolytic enzyme of the host cell.

“Processing” may take place N-terminally and, where appropriate, also C-terminally of the sequence of mature, processed protein (target protein).

Although a “homologous” target protein is originally expressed in the host used according to the invention and is thus a protein endogenous to the host, it is secreted by the host cells, owing to transformation of said host with an expression construct of the invention.

A “heterologous” target protein is originally not expressed in the host used according to the invention and is thus not an endogenous host protein, but is secreted by the host cells, owing to transformation of said host with the expression construct of the invention.

A “shuttle peptide” is part of a “shuttle peptide construct” processable in the host cell used according to the invention. It forms said shuttle peptide construct together with one or more processable regulatory peptide fragments C- and/or N-terminally, preferably N-terminally, linked therewith, such as signal sequences, leader sequences. In contrast to the signal peptide, for example, the shuttle peptide is a polypeptide secreted by the host cell. Processing of the regulatory elements is preferably carried out intracellularly. The shuttle peptide remains secretable, even when it is, preferably C-terminally, processably fused to a target protein. This C-terminal processing, i.e. removal of the target protein proteolytically in the course of secretion, is preferably carried out, for example, during passage through the envelope of the host cell, or in the extracellular space, for example in the surrounding culture medium, by endogenous cellular proteases.

An “expression construct” or an “expression cassette” according to the present invention comprises, operatively linked to the coding nucleic acid sequence of a processable shuttle peptide construct as defined above, the start and termination signals for transcription and, where appropriate, translation, which are required for controlling expression in a special host system such as, in particular, yeast cells. The expression construct in particular encompasses binding sites for transcription factors. There is comprised a constitutive or inducible, native or heterologous, natural or synthetic promoter operable in the host cell 5′ upstream of the coding sequence. The expression construct moreover encompasses a number of restriction enzyme cleavage sites such as, for example, those for inserting said construct into an expression vector. In addition, the expression construct may encompass a selectable marker gene.

An “expression vector” describes a construct obtainable by introducing an expression cassette of the invention into a replicon, for example into a plasmid, cosmid or virus. A vector of this kind is capable of autonomous replication or of integrating into the host genome and comprises the required control sequences for controlling transcription and, where appropriate, translation of the nucleic acid sequences coding according to the invention for a processable shuttle peptide construct as defined above.

b) Preferred Embodiments

The invention firstly relates to an expression construct which comprises the nucleic acid sequence coding for a shuttle peptide construct which is processable by yeast cells, has the formula
(Sig-SP),
and comprises in 5′-3′ orientation the nucleic acid sequences coding for
a) a signal peptide (Sig) processably linked to
b) at least one shuttle peptide (SP) secretable by said yeast cells;
   and, where appropriate, one or more nucleic acid sequences promoting processing and/or secretion and located 5′- or 3′-terminally of the coding signal peptide sequence.

The sequences coding for SP and Sig are within the same reading frame and, moreover, a processable sequence between the C terminus of Sig and the N terminus of SP is formed during translation. An example of said processable sequence is an artificially introduced, proteolytically cleavable natural or synthetic adaptor sequence which is, however, preferably part of the C terminus of Sig or the N terminus of SP. The adaptor sequence may be processed in such a way that the cleaved sequence may be found entirely or partially at the C terminus of Sig or the N terminus of SP. The latter is possible as long as this has no substantial adverse effect on, and in particular does not prevent, the secretability of SP.

The invention in particular relates to those expression constructs which code for a processable shuttle peptide construct derived from a polypeptide processed by yeasts in the broadest sense. Said yeasts are in particular those selected from among ascomycetes. Preference is given to yeasts selected from those of the class of Archiascomycetes, the order of Schizosaccharomycetales, and yeasts are particularly preferably selected from among those of the genus Schizosaccharomyces, such as S. pombe. Although there are data which indicate that Minus cells also secrete P factor, preference is given to using the strain matching the mating factor (pheromone) (i.e. Plus cells for the Plus factor (P factor) and Minus cells for the Minus factor (M factor).

The processable shuttle peptide construct is derived, in particular, from a pheromone pre-protein of a yeast, said pheromone being produced from said pre-protein by N- and C-terminal processing. The pheromone preferably has N-terminally a polypeptide removable by processing, which encompasses in particular the elements required for processing and/or secreting the pre-protein, such as signal peptide and, where appropriate, leader peptide, and also the required protease cleavage sites.

Fungal pheromones are known and have been described, for example, both for basidiomycetes such as Ustilago maydis (Urban, M., Kahmann, R. and Bolker, M. (1996) The biallelic a mating type locus of Ustilago maydis: remnants of an additional pheromone gene indicate evolution from a multiallelic ancestor (Mol Gen Genet 250(4):414-420)) or Coprinopsis cinera (Halsall, J. R., Milner, M. J. and Casselton, L. A. (2000) Three subfamilies of pheromone and receptor genes generate multiple B mating specificities in the mushroom Coprineus cinereus (Genetics 154(3):1115-1123)) and for ascomycetes such as Schizosaccharomycespombe (Imai, Y. and Yamamoto, M. (1995) The fission yeast mating pheromone P-factor: its molecular structure, gene structure, and ability to induce gene expression and G1 arrest in the mating partner (Genes Dev 8(3):328-338), Davey, J. (1992) Mating pheromones of the fission yeast Schizosaccharomyces pombe: purification and structural characterization of M-factor and isolation and analysis of two genes encoding the pheromone (EMBO J 11(3):951-960)), Saccharomyces cerevisiae (Michaelis, S. and Herskowitz, I. (1988) The a-factor pheromone of Saccharomyces cerevisiae is essential for mating (Mol Cell Biol 8(3):1309-1318), Kurjan, J. and Herskowitz, I. (1982) Structure of a yeast pheromone gene (MF-alpha): a putative alpha-factor precursor contains four tandem copies of mature alpha-factor (Cell 30(3):933-943)), Kluyveromyces delphensis (Wong, S., Fares, M. A., Zimmermann, W., Butler, G. and Wolfe, K. H. (2003) Evidence from comparative genomics for a complete sexual cycle in the ‘asexual’ pathogenic yeast Candida glabrata (Genome Biol 4(2)R10)) and Saccharomyces kluyveri (Egel-Mitani, M. and Hansen, M. T. (1987) Nucleotide sequence of the gene encoding the Saccharomyces kluyveri alpha mating pheromone (Nucleic Acids Res 15(15)6303)).

Pheromones suitable according to the invention are relatively small peptides (such as, for example, from 5 to 40 or from 8 to 30 amino acids). Their primary sequence usually exhibits no significant homology. They are formed as pre-proteins, proteolytically processed and released into the culture medium.

Examples of particularly suitable pheromones or corresponding pre-proteins are the “P factor” and “M factor” and their pre-proteins from S. pombe. (cf. Imai, Y. and Yamamoto, M. (1995) The fission yeast mating pheromone P-factor: its molecular structure, gene structure, and ability to induce gene expression and G1 arrest in the mating partner (Genes Dev 8(3):328-338), Davey, J. (1992) Mating pheromones of the fission yeast Schizosaccharomyces pombe: purification and structural characterization of M-factor and isolation and analysis of two genes encoding the pheromone (EMBO J 11(3):951-960), Kjaerulff, S., Davey, J. and Nielsen, O. (1994) Analysis of the structural genes encoding M-factor in the fission yeast Schizosaccharomyces pombe: identification of a third gene, mfm3 (Mol Cell Biol 14(6)3895-3905)).

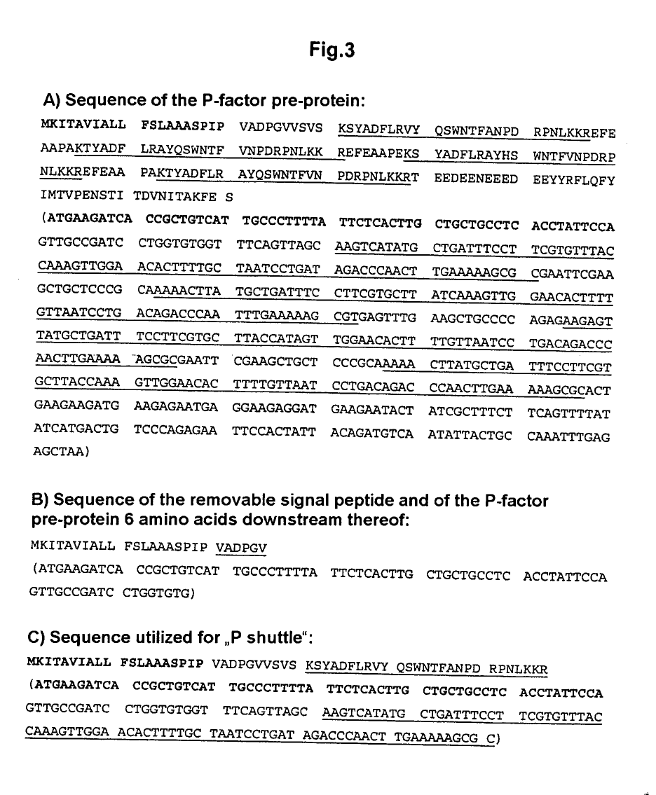

The P-factor pre-protein, for example, has a DNA sequence according to SEQ ID NO: 9 and a protein sequence according to SEQ ID NO:10. The pre-protein encompasses an N-terminal signal peptide sequence bridged with four consecutive pheromone peptide sequences separable by processing (cf. FIG. 3).

In preferred constructs of the invention, the processable shuttle peptide construct is designed so as to comprise a signal polypeptide (Sig) which is processably linked to the N-terminal end of a C-terminally processable pheromone polypeptide (Pher).

The signal polypeptide encompasses in particular the proteolytically removable native signal polypeptide (e.g. SEQ ID NO:4 encoded by SEQ ID NO:3) of the pheromone pre-protein or is identical thereto.

Preference is furthermore given to the C-terminally processed pheromone polypeptide encompassing a C-terminal protease cleavage site.

The expression construct preferably furthermore encompasses the nucleic acid sequence coding for a homologous or heterologous target protein (Targ) processably linked to the C terminus of the shuttle peptide construct (Sig-SP).

The invention preferably relates to expression constructs of the abovementioned type, encompassing the nucleic acid sequence coding for a fusion protein which is processable by yeast cells and has the formula
Sig-L1ₙ-Pher-L2ₘ-Targ
in which
Sig, Pher and Targ are as defined above,
L1 and L2 are processable linkers or adaptor sequences and
n and m are independently of one another 0 or 1. Preferably, however, n is 1 and m is 0.

L1 and L2 may be natural or synthetic linkers. They encompass at least one proteolytic processable peptide sequence. Where appropriate, other effector functions promoting, for example, processing, secretion, transcription and/or translation may be associated with L1 and/or L2.

Particular preference is given to expression constructs wherein the nucleic acid sequence coding for the processable shuttle peptide construct encompasses a signal polypeptide (Sig)-encoding sequence according to SEQ ID NO: 3 or a functional equivalent thereof, operatively linked to the nucleic acid sequence according to SEQ ID NO:5 coding for the mature P-factor pheromone (Pher), or a functional equivalent thereof.

Preferably, the linker L2 is not present. In contrast, preference is given to providing the linker L1 which encompasses the sequence coding for a polypeptide according to amino acid residues 21 to 30 in SEQ ID NO:10. L1 here bridges the signal polypeptide with the first pheromone building block (positions 31 to 57 in SEQ ID NO:10) of the prehormone. The C-terminal end of L1 corresponds to a recognition sequence of the protease required for proteolytic processing.

In a particularly preferred embodiment, the nucleic acid sequence coding for the processable shuttle peptide construct encompasses a sequence according to SEQ ID NO:1.

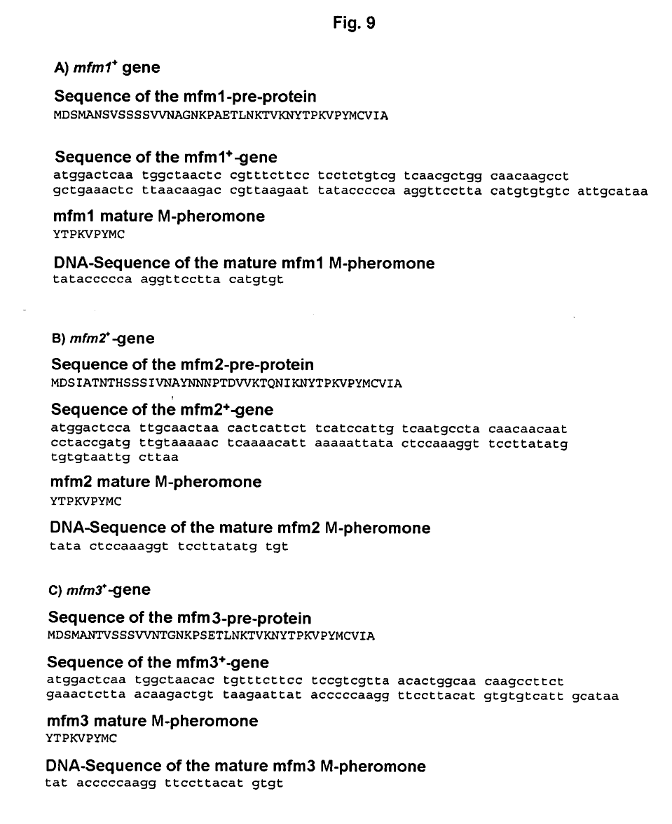

The same procedure may in principle also be used with the aid of the M factor, the second pheromone present in S. pombe, and be applied to expressing any homologous and heterologous target proteins. There are three genomic genes (mfm1⁺, SEQ ID NO:42; mfm2⁺, SEQ ID NO: 45; and mfm3⁺, SEQ ID NO:48) which in each case encode the M factor, the pheromone of cells having the Minus mating type. First a preprotein (SEQ ID NO: 43, 46 and 49) is produced from each gene, which is processed in the course of secretion. Finally, the M factor (YTPKVPYMC; SEQ ID NO:51) encoded by SEQ ID NO: 44, 47 and 50, respectively) is released as mature pheromone into the medium (cf. FIG. 9).

Further shuttle peptide constructs suitable according to the invention might therefore be derived from the coding sequences according to SEQ ID NO:42, 45 or 48 which code for M-factor signal peptide functionally linked to an M-factor pheromone. Nonlimiting examples of corresponding coding shuttle peptide sequences encompass, for example, nucleotide residues 1 to 117 according to SEQ ID NO:42; nucleotide residues 1 to 123 according to SEQ ID NO:45; or nucleotide residues 1 to 114 according to SEQ ID NO:48; or functionally equivalent constructs derived therefrom which control secretion and processing of the M-factor pheromone and of a homologous or heterologous target protein C-terminally and proteolytically removably linked to said pheromone. Functional equivalents may comprise the sequence sections located 5′ upstream of the coding sequence of the mature M factor (SEQ ID NO:44, 47 or 50) in an unchanged or modified (e.g. by deletion of single or multiple nucleic acid residues) form and thus code for a shuttle peptide which has an altered amino acid sequence and which functionally links the mature M-factor peptide sequence to a, for example C-terminally truncated, signal sequence section.

A target protein (Targ) expressed according to the invention may be derived from any prokaryotic or eukaryotic organism, in particular humans, animals or yeasts, as long as it can be expressed, secreted and processed by the host cell in the manner according to the invention as part of a fusion protein with the shuttle peptide (SP). The secreted and processed product may be therapeutically useful or may have other advantageous applicable properties. Examples of therapeutically useful proteins which may be mentioned here are immunoglobulins, peptide hormones, growth factors, lymphokines, protease inhibitors and the like. Examples of target proteins having other properties with interesting application, which may be mentioned here, are in particular hydrophobins.

In a particularly preferred embodiment of the invention, the target protein is a hydrophobin, in particular a class I hydrophobin.

Typical hydrophobins are relatively small (100±25 amino acids) moderately hydrophobic proteins having a conserved motif of 8 cysteins (X₂₋₃₈-C-X₅₋₉-C-C-X₁₁₋₃₉-C-X₈₋₂₃-C-X₅₋₉-C-C-X₆₋₁₈-C-X₂₋₁₃). Hydrophobins may assemble at hydrophilic-hydrophobic interfaces to give protein films. Such aggregates of class I hydrophobins are insoluble in SDS, while aggregates of class II hydrophobins are soluble in SDS (Wessels, J. G. H. (1997) Hydrophobins: Proteins that change the nature of the fungal surface. Adv Microb Physiol 38:1-45).

Hydrophobins usable according to the invention are in particular derived from fungi, for example from ascomycetes such as those of the genus Aspergillus, in particular A. nidulans.

Usable hydrophobins are also known from the prior art mentioned above and are not limited to those from fungi.

Nonlimiting examples of usable hydrophobins are selected from among SEQ ID NO: 14 (DewA), SEQ ID NO:19 (RdlA) SEQ ID NO:20 (RdlB) SEQ ID NO:21 (HYP1) SEQ ID NO:22 (HYP4) and SEQ ID NO:56 (RodA).

p52750 (DewA)
MRFIVSLLAF TAAATATALP ASAAKNAKLA TSAAFAKQAE
GTTCNVGSIA CCNSPAETNN DSLLSGLLGA GLLNGLSGNT
GSACAKASLI DQLGLLALVD HTEEGPVCKN IVACCPEGTT
NCVAVDNAGA GTKAE
q91190 (RdIA)
MLKKAMVAAA AAASVIGMSA AAAPQALAIG DDNGPAVANG
NGAESAFGNS ATKGDMSPQLSLVEGTLNKP CLGVEDVNVA
VINLVPIQDI NVLADDLNQQ CADNSTQAKR DGALSHVLED
LSVLSANGEG R
q934f8 (RdIB)
MIKKVVAYAA IAASVMGASA AAAPQAMAIG DDSGPVSANG
NGASQYFGNS MTTGNMSPQM ALIQGSFNKP CIAVSDIPVS
VIGLVPIQDL NVLGDDMNQQ CAENSTQAKR DGALAHLLED
VSILSSNGEG GKG
HYP1_AGABI (P49072)
MISRVLVAAL VALPALVTAT PAPGKPKASS QCDVGEIHCC
DTQQTPDHTS AAASGLLGVP INLGAFLGFD CTPISVLGVG
GNNCAAQPVC CTGNQFTALI NALDCSPVNV NL
HYP4_AGABI (043122)
MVSTFITVAK TLLVALLFVN INIVVGTATT GKHCSTGPIE
CCKQVMDSKS PQATELLTKN GLGLGVLAGV KGLVGANCSP
ITAIGIGSGS QCSGQTVCCQ NNNFNGVVAI CTPINANV
RodA
LPPAHDSQFA GNGVGNKGNS NVKFPVPENV TVKQASDKCG
DQAQLSCCNK ATYAGDTTTV DEGLLSGALS GLIGAGSGAE
GLGLFDQCSK LDVAVLIGIQ DLVNQKCKQN IACCQNSPSS
ADGNLIGVGL PCVALGSIL

The RodA protein is, together with the DewA protein, part of the outer spore capsule of A. nidulans.

The invention moreover relates to expression vectors which encompass an expression construct as defined above in operative linkage to at least one regulatory nucleic acid sequence.

The invention also relates to recombinant microorganisms comprising at least one expression vector or an expression construct as defined above, where appropriate stably integrated into the host genome.

A “recombinant microorganism in accordance with the present invention encompasses at least one expression vector of the invention or an expression construct of the invention and is derived from yeasts in the broadest sense. Said yeasts are in particular derived from ascomycetes. Preferred yeasts are selected from the class of Archiascomycetes, the order Schizosaccharomycetales, and are particularly preferably selected from among yeasts of the genus Schizosaccharomyces such as S. pombe.

The invention further relates to shuttle peptide constructs which can be processed by yeast cells and are derived from a yeast pheromone pre-protein, the pheromone being derivable from said pre-protein by N- and C-terminal processing and being secretable.

Preference is given to those shuttle peptide constructs which comprise a signal polypeptide N-terminally processably linked to the C-terminally processed pheromone polypeptide.

Said signal polypeptide is preferably the proteolytically removable native signal polypeptide of the pheromone pre-protein, and the C-terminally processed pheromone polypeptide encompasses the C-terminal protease cleavage site.

Preferred shuttle peptide constructs are derived here from pheromone pre-proteins of yeasts, in particular pre-proteins of the S. pombe factors P and M. Particularly preferred shuttle peptides encompass an amino acid sequence according to SEQ ID NO:2 or a functional equivalent thereof.

The invention further relates to a method for recombinant preparation of a target protein, which comprises culturing a recombinant microorganism as defined above, expressing the nucleic acid sequence encoding said target protein and isolating the target protein secreted into the culture medium, such as, for example, a hydrophobin as defined above.

The invention furthermore relates to nucleic acids coding for a shuttle peptide construct as defined above; and to nucleic acids coding for a fusion protein as defined above which can be processed and by yeast cells and encompasses a target protein.

The invention also relates to hydrophobins obtainable by a method of the invention.

Finally, the invention relates to the use of such a hydrophobin for surface treatment, which comprises treating in particular the surface of objects selected from among glass, fibers, fabrics, leather, painted objects such as, for example, motor vehicle bodies, films, facades.

The invention also relates to the use of hydrophobins for treating the surfaces of fibers, fabrics and leather.

c) Further Embodiments of the Invention

c1) Polypeptides/Proteins

The invention also comprises “functional equivalents” of the specifically disclosed or used polypeptide/proteins. This applies both to the intermediately produced fusion proteins and to components thereof, i.e. target proteins (Targ), shuttle peptides (SP), such as pheromones (Pher), but also to signal peptides (Sig) and linkers. The generic term used for polypeptide/protein will only be “polypeptide” hereinbelow.

“Functional equivalents” or analogs of the specifically disclosed polypeptides are, within the scope of the present invention, polypeptides differing therefrom which furthermore have the desired biological activity. Analogous shuttle peptides should furthermore be suitable for controlling secretion and processing of the target protein. Correspondingly, the functional equivalents of components of the shuttle peptide, such as signal polypeptide, pheromone, linker, are also intended to have furthermore the properties required for an effective secretion and processing of the fusion protein with release of the target protein.

“Functional equivalents” of inventive polypeptides, such as target proteins, shuttle peptides, may comprise at the C and/or N terminals in particular remnants of natural linker or adaptor sequences, which result from proteolytic cleavage.

“Functional equivalents” mean according to the invention in particular mutant proteins which have in at least one of the sequence positions of the abovementioned specific sequences an amino acid different from the one specifically mentioned, but which have nevertheless one of the abovementioned biological activities. “Functional equivalents” thus encompass the mutant proteins obtainable by one or more amino acid additions, substitutions (cf. examples in the table below), deletions and/or inversions, it being possible for said modifications to occur in any sequence position, as long as they result in a mutant protein having the property profile of the invention.

Examples of residues suitable for amino acid substitutions are:

| Original residue | Substitution examples |
|---|---|
| Ala | Ser |
| Arg | Lys |
| Asn | Gln; His |
| Asp | Glu |
| Cys | Ser |
| Gln | Asn |
| Glu | Asp |
| Gly | Pro |
| His | Asn; Gln |
| Ile | Leu; Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

There is functional equivalence in particular also when the mutant and the unaltered polypeptide have qualitatively matching activity patterns. This means, for example, that modified shuttle peptides express or secrete the same target protein in the same host with higher or lower efficiency; or that modified target proteins have an increased or reduced pharmacological action or modified applicable property.

“Functional equivalents” in the above sense also encompass precursors of the polypeptides described and also functional derivatives and salts of said polypeptides. The expression “salts” means both salts of carboxyl groups and acid addition salts of amino groups of the protein molecules of the invention. Salts of carboxyl groups may be prepared in a manner known per se and encompass inorganic salts such as, for example, sodium, calcium, ammonium, iron and zinc salts and also salts with organic bases such as, for example, amines, such as triethanolamine, arginine, lysine, piperidine and the like. The invention likewise relates to acid addition salts such as, for example, salts with mineral acids such as hydrochloric acid or sulfuric acid and salts with organic acids such as acetic acid and oxalic acid.

“Functional derivatives” of polypeptides of the invention may likewise be prepared on functional amino acid side groups or on their N- or C-terminal ends with the aid of known techniques. Derivatives of this kind encompass, for example, aliphatic esters of carboxylic acid groups, amides of carboxylic acid groups, obtainable by reaction with ammonia or with a primary or secondary amine; N-acyl derivatives of free amino groups, prepared by reaction with acyl groups; or O-acyl derivatives of free hydroxy groups, prepared by reaction with acyl groups.

“Functional equivalents” also encompass, of course, polypeptides obtainable from organisms different from those specifically mentioned and also naturally occuring variants. For example, it is possible to establish by sequence comparison regions of homologous sequence regions and to determine equivalent enzymes following the specific guidelines of the invention.

“Functional equivalents” also encompass fragments, preferably individual domains or sequence motifs, of the polypeptides of the invention, which have the desired biological function, for example.

“Functional equivalents” are moreover fusion proteins which have one of the above-mentioned polypeptide sequences or functional equivalents derived therefrom and at least one further heterologous sequence functionally different therefrom in functional N- or C-terminal linkage (i.e. with mutual negligible functional impairment of the fusion protein parts). Nonlimiting examples of heterologous sequences of this kind are, for example, signal peptides, enzymes, immunoglobulins, surface antigens, receptors or receptor ligands.

“Functional equivalents” also encompassed by the invention are homologs of the specifically mentioned polypeptides. Said homologs are at least 60%, preferably at least 75%, in particular at least 85%, such as, for example, 90%, 95% or 99%, homologous to any of the specifically disclosed sequences, calculated according to the algorithm of Pearson and Lipman, Proc. Natl. Acad, Sci. (USA) 85(8), 1988, 2444-2448. A percentage homology of a homologous polypeptide of the invention means in particular percentage identity of the amino acid residues based on the total length of any of the amino acid sequences specifically described herein.

In the case of a possible protein glycosylation, equivalents of the invention encompass polypeptides in deglycosylated or glycosylated form and modified forms obtainable by altering the glycosylation pattern.

Homologs of the proteins or polypeptides of the invention may be generated in a manner known per se by mutagenesis, for example by point mutagenesis or truncation of the protein.

c2) Nucleic Acid Sequences:

Nucleic acid sequences of the invention, in particular those coding for any of the above polypeptides and their functional equivalents, encompass single- and double-stranded DNA and RNA sequences such as, for example, also cDNA and mRNA.

All nucleic acid sequences mentioned herein either are of natural origin or can be prepared in a manner known per se by chemical synthesis of nucleotide building blocks such as, for example, by fragment condensation of individual overlapping, complementary nucleic acid building blocks.

The chemical synthesis of oligonucleotides may be carried out in a manner known per se, for example according to the phosphoramidite method (Voet, Voet, 2nd edition, Wiley Press New York, pages 896-897). The assembly of synthetic oligonucleotides and filling-in of gaps with the aid of the Klenow fragment of DNA polymerase and ligation reactions and also general cloning methods are described in Sambrook et al. (1989), Molecular Cloning: A laboratory manual, Cold Spring Harbor Laboratory Press.

The invention also relates to nucleic acid sequences coding for any of the above polypeptides and their functional equivalents, which are accessible by using artificial nucleotide analogs, for example.

The invention relates both to isolated nucleic acid molecules coding for polypeptides of the invention or biologically active sections thereof and to nucleic acid fragments suitable, for example, for use as hybridization probes or primers for identifying or amplifying coding nucleic acids of the invention.

The nucleic acid molecules of the invention may moreover comprise untranslated sequences of the 3′ and/or 5′ end of the coding gene region.

An “isolated” nucleic acid molecule is removed from other nucleic acid molecules present in the natural source of said nucleic acid and may, in addition, be essentially free of other cellular material or culture medium when prepared by recombinant techniques, or free of chemical precursors or other chemicals, when synthesized chemically.

A nucleic acid molecule of the invention may be isolated by means of molecular-biological standard techniques and the sequence information provided according to the invention. For example, cDNA may be isolated from a suitable cDNA bank by using any of the specifically disclosed complete sequences or a section thereof as hybridization probe and standard hybridization techniques (as described, for example, in Sambrook, J., Fritsch, E. F. and Maniatis, T. Molecular Cloning: A Laboratory Manual. 2^{nd} edition, Cold Spring Harbor Laboratory, Cold Spring Harbor Laboratory Press, Cold Spring Harbor, N.Y., 1989). In addition, it is possible to isolate a nucleic acid molecule encompassing any of the disclosed sequences or a section thereof by polymerase chain reaction, using the oligonucleotide primers produced on the basis of said sequence. The nucleic acid amplified in this way may be cloned into a suitable vector and characterized by DNA sequence analysis.

The invention furthermore encompasses the nucleic acid molecules complementary to the specifically described nucleotide sequences or a section thereof.

Said nucleotide sequences enable probes and primers to be generated which may be used for identifying and/or cloning homologous sequences in other cell types and organisms. Such probes or primers usually encompass a nucleotide sequence region which hybridizes under stringent conditions to at least about 12, preferably to at least about 25, such as, for example, about 40, 50 or 75, consecutive nucleotides of a sense strand of a nucleic acid sequence of the invention or of a corresponding antisense strand.

Further nucleic acid sequences of the invention are derived from the specifically disclosed sequences and differ therefrom by addition, substitution, insertion or deletion of single or multiple nucleotides, but still code for polypeptides having the desired property profile.

The invention also encompasses those nucleic acid sequences which encompass “silent” mutations or which have been modified according to the codon usage of a special source or host organism, in comparison with a specifically mentioned sequence, as well as naturally occurring variants thereof such as, for example, splice variants or allele variants. The invention also relates to sequences obtainable by conservative nucleotide substitutions (replacing the amino acid in question with an amino acid of identical charge, size, polarity and/or solubility).

The invention also relates to the molecules derived from the specifically disclosed nucleic acids by sequence polymorphisms. These genetic polymorphisms may exist between individuals within a population due to natural variation. These natural variations usually cause a variance of from 1 to 5% in the nucleotide sequence of a gene.

The invention furthermore also encompasses nucleic acid sequences which hybridize with abovementioned coding sequences or which are complementary thereto. These polynucleotides can be found when screening genomic or cDNA banks and, where appropriate, be amplified therefrom by means of PCR using suitable primers and subsequently be isolated using suitable probes, for example.

The property of being able to “hybridize” to polynucleotides means the ability of a poly- or oligonucleotide to bind under stringent conditions to a nearly complementary sequence, while unspecific bondings between noncomplementary partners do not occur under these conditions. For this purpose, the sequences should be 70-100%, preferably 90-100%, complementary. The property of complementary sequences of being able to specifically bind to one another is utilized, for example, in the Northern or Southern blot technique or for primer binding in PCR or RT-PCR. Usually oligonucleotides of 30 or more base pairs in length are used for this purpose. Stringent conditions mean, for example in the Northern blot technique, the use of a washing solution at 50-70° C., preferably 60-65° C., for example 0.1×SSC buffer containing 0.1% SDS (20×SSC: 3M NaCl, 0.3M sodium citrate, pH 7.0) for eluting unspecifically hybridized cDNA probes or oligonucleotides. This means, as mentioned above, only highly complementary nucleic acids remain bound to one another. Setting stringent conditions is known to the skilled worker and is described, for example, in Ausubel et al., Current Protocols in Molecular Biology, John Wiley & Sons, N.Y. (1989), 6.3.1-6.3.6.

c3) Expression Constructs and Vectors:

The invention moreover relates to expression constructs comprising a nucleic acid sequence coding for a polypeptide to be expressed according to the invention under the genetic control of regulatory nucleic acid sequences; and to vectors encompassing at least one of said expression constructs.

Such constructs of the invention preferably encompass a promoter 5′ upstream of the particular coding sequence and a terminator sequence 3′ downstream and, where appropriate, further common regulatory elements, in each case operatively linked to the coding sequence.

“Operative linkage” means the sequential arrangement of promoter, coding sequence, terminator and, where appropriate, other regulatory elements such that each of the regulatory elements can fulfill, as required, its function in expressing the coding sequence.

Examples of operatively linkable sequences are targeting sequences and also enhancers, polyadenylation signals and the like. Other regulatory elements encompass selectable markers, amplification signals, origins of replication and the like. Suitable regulatory sequences are described, for example, in Goeddel, Gene Expression Technology: Methods in Enzymology 185, Academic Press, San Diego, Calif. (1990).

The gene construct may comprise one or more copies of the coding nucleic acid sequences.

Examples of usable promoters are the yeast promoters ADC1, MFalpha , AC, P-60, CYC5, GAPDH, nmt1, nmt41 and nmt81.

Examples of suitable promoters for the yeast S. pombe, which may be mentioned, are: nmt1, nmt41, nmt81, adh1, fbp1, SV40 or CaMV. Further information under (http://pingu.salk.edu/˜forsburg/vectors.html#exp). The promoters differ with respect to their rate of transcription. The selection depends on the desired level of expression. This applies to other yeasts accordingly.

Suitable yeast promoters are described, for example, in the published US patent application 2003/0077831 to which reference is expressly made hereby.

It is also useful to use inducible promoters such as, for example, light- and, in particular, temperature-inducible promoters.

The regulatory sequences mentioned are intended to make possible targeted expression of said nucleic acid sequences and of protein expression. Depending on the host organism, this may mean, for example, that the gene is expressed or overexpressed only after induction or that it is expressed and/or overexpressed immediately.

In this connection, the regulatory sequences or factors may preferably influence expression in a positive way and thereby increase or reduce the latter. Thus, the regulatory elements may advantageously be enhanced at the transcriptional level by using strong transcription signals such as promoters and/or “enhancers”. Apart from this, however, it is also possible to enhance translation by improving, for example, mRNA stability.

An expression cassette is prepared by fusing a suitable promoter to a suitable coding nucleotide sequence and to a terminator signal or polyadenylation signal. For this purpose, common recombination and cloning techniques are used, such as those described, for example, in T. Maniatis, E. F. Fritsch and J. Sambrook, Molecular Cloning: A Laboratory Manual, Cold Spring Harbor Laboratory, Cold Spring Harbor, N.Y. (1982) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

The recombinant nucleic acid construct or gene construct is expressed in a suitable host organism by inserting it advantageously into a host-specific vector which makes optimal expression of the genes in the host possible. Vectors are well known to the skilled worker and can be found, for example, in “Cloning Vectors” (Pouwels P. H. et al., eds., Elsevier, Amsterdam-New York-Oxford, 1985). Vectors mean, apart from plasmids, also any other vectors known to the skilled worker, such as, for example, phages, viruses, such as SV40, CMV, baculovirus and adenovirus, transposons, IS elements, phasmids, cosmids and linear or circular DNA. These vectors may be replicated autonomously in the host organism or chromosomally.

Examples of expression vectors suitable according to the invention, which may in particular be mentioned here, are constructs suitable for the yeast S. pombe (see e.g.: (http://pingu.salk.edu/˜forsburg/vectors.html#exp).

Other Examples are:

pART1 (McLeod, M., Stein, M., and Beach, D. (1987) The product of the mei3+ gene expressed under control of the mating type locus, induces meiosis and sporulation in fission yeast. EMBO J. 6:729-736

pCHY21 (Hoffman, C. S. and Winston, F. (1991). Glucose repression of transcription of the schizosaccharomyces pombe fbp1 gene occurs by a camp signaling pathway. Genes Dev. 5:561-571)

REP1 ,REP3, REP4 (Maundrell, K. (1990). nmt1 of fission yeast: a highly transcribed gene completely repressed by thiamine. J. Biol. Chem. 265:10857-10864)

REP41, REP42, REP81, REP82 (Basi, G., Schmid, E. and Maundrell, K. (1993). TATA box mutations in the Schizosaccharomyces pombe nmt1 promoter affect transcription efficiency but not the transcription start point or thiamine repressibility. Gene 123:131-136)

Yeast expression vectors for expression in the yeast S. cerevisiae, such as pYEpSec1 (Baldari et al., (1987) Embo J. 6:229-234), pMFa (Kurjan and Herskowitz (1982) Cell 30:933-943), pJRY88 (Schultz et al. (1987) Gene 54:113-123) and pYES2 (Invitrogen Corporation, San Diego, Calif.). Vectors and methods for constructing vectors, which are suitable for use in other fungi such as filamentous fungi, include those which are described in detail in: van den Hondel, C.A.M.J.J. & Punt, P.J. (1991) “Gene transfer systems and vector development for filamentous fungi, in: Applied Molecular Genetics of Fungi, J. F. Peberdy et al., eds., pp. 1-28, Cambridge University Press: Cambridge.

Other suitable expression systems are described in chapters 16 and 17 of Sambrook, J., Fritsch, E. F. and Maniatis, T., Molecular cloning: A Laboratory Manual, 2^{nd} edition, Cold Spring Harbor Laboratory, Cold Spring Harbor Laboratory Press, Cold Spring Harbor, N.Y., 1989.

c4) Recombinant Microorganisms:

It is possible with the aid of the vectors of the invention, to prepare recombinant microorganisms which have been transformed, for example, with at least one vector of the invention and which may be used for producing the polypeptides of the invention.

Advantageously, the above-described recombinant constructs of the invention are introduced into a suitable host system and expressed. Preferably, this involves using familiar cloning and transfection methods known to the skilled worker, such as, for example, coprecipitation, protoplast fusion, electroporation, retroviral transfection and the like, in order to make the nucleic acids mentioned be expressed in the particular expression system. Suitable systems are described, for example, in Current Protocols in Molecular Biology, F. Ausubel et al., eds., Wiley Interscience, New York 1997, or Sambrook et al. Molecular Cloning: A Laboratory Manual. 2^{nd} ed., Cold Spring Harbor Laboratory, Cold Spring Harbor Laboratory Press, Cold Spring Harbor, N.Y., 1989.

Suitable host organisms are in principle any organisms which enable nucleic acids of the invention, their allelic variants, their functional equivalents or derivatives to be expressed. Preferred host organisms are yeasts.

Methods for introducing exogenous DNA into yeast cells are known from the prior art. Said introduction may be carried out, for example, by spheroplast transformation according to Hinnen et al. (1978, Proc. Natl. Acad. Sci., USA, 75: 1919-1935). Chemical transformation methods can be found, for example for S. pombe, in Alfa et al. (Alfa, C., Fantes, P., Hyams, J., McLeod, M. and Warbrick, E. (1993) Experiments with fission yeast. Cold Spring Harbor Laboratory Press, New York) or, for S. cerevisiae, in Kaiser et al. (Kaiser, C., Michaelis, S. and Mitchell, A. (1994) Methods in Yeast Genetics. Cold Spring Harbor Laboratory Press, New York).

Auxotrophic markers are frequently utilized in yeasts to select transformants. In this case, the strain to be transformed lacks a protein required for producing particular metabolic products. The corresponding active protein is introduced into the cell by the utilized vector. Frequently utilized markers are genes of uracil, leucine, histidine or tryptophan biosynthesis, for example.

Successfully transformed organisms may be selected via marker genes which are also included in the vector or in the expression cassette. Examples of such marker genes are genes for antibiotic resistance and for enzymes catalyzing a color-producing reaction which causes the transformed cell to be stained. The latter may then be selected by means of automated cell sorting. Microorganisms which have been transformed successfully with a vector and which carry an appropriate antibiotic resistance gene (e.g. G418 or hygromycin) can be selected via corresponding antibiotic-containing media or nutrient beds. Marker proteins exposed on the cell surface may be utilized for selection by means of affinity chromatography.

The combination of the host organisms and the appropriate vectors for said organisms, such as plasmids, viruses or phages, for example plasmids containing the RNA polymerase/promoter system, the phages 8 or: or other temperate phages or transposons, and/or other advantageous regulatory sequences constitutes an expression system.

c5) Recombinant Preparation of Target Proteins

The invention furthermore relates to methods for recombinant preparation of a target protein as defined above.

The recombinant microorganism may be cultured and fermented according to known methods. Suitable culturing conditions are described in detail, for example for S. pombe in Alfa et al. (Alfa, C., Fantes, P., Hyams, J., McLeod, M. and Warbrick, E. (1993) Experiments with fission yeast. Cold Spring Harbor Laboratory Press, New York) and Gutz et al. (Gutz, H., Heslot, H., Leupold, U. and Loprieno, U. (1974) Schizosaccharomyces pombe. In: Handbook of Genetics 1, pp 395-446, Plenum Press, New York) or, for S. cerevisiae, in Kaiser et al. (Kaiser, C., Michaelis, S. and Mitchell, A. (1994) Methods in Yeast Genetics. Cold Spring Harbor Laboratory Press, New York).

If the target protein is secreted into the culture supernatant, the cells are removed from the latter and the target protein is obtained from the supernatant according to known protein isolation methods.

The target protein may be purified using known, chromatographic methods such as molecular-sieve chromatography (gel filtration), ion exchange chromatography such as Q-Sepharose chromatography, and hydrophobic chromatography, and using other common methods such as ultrafiltration, crystallization, salting out, dialysis and native gel electrophoresis. Suitable methods are described, for example, in Cooper, F. G., Biochemische Arbeitsmethoden [Original title: The Tools of Biochemistry], Verlag Walter de Gruyter, Berlin, New York or in Scopes, R., Protein Purification, Springer Verlag, New York, Heidelberg, Berlin.

Isolation of the recombinant protein may also be facilitated by using vector systems coding for altered polypeptides or fusion proteins which simplify purification. Examples of suitable modifications of this kind are “Tags” acting as anchors, such as, for example, the modification known as hexa-histidine anchor or epitopes which can be recognised as antigens by antibodies (described, for example, in Harlow, E. and Lane, D., 1988, Antibodies: A Laboratory Manual. Cold Spring Harbor (N.Y.) Press). These anchors may be used for attaching the proteins to a solid support such as, for example, a polymer matrix which can, for example, be packed into a chromatography column, or may be used on a microtiter plate or another support.

These anchors may at the same time also be used for recognition of the proteins. Moreover, the proteins may be recognised by using conventional markers such as fluorescent dyes, enzyme markers forming a detectable reaction product after reaction with a substrate, or radioactive labels, alone or in combination with said anchors for derivatizing the proteins.

c6) Surface Treatment with Hydrophobin

The treatment of surfaces with hydrophobins in order to alter, for example hydrophobize or hydrophilize, the surface properties is known in principle, and is now substantially simplified by the invention whose recombinant preparation of the hydrophobins provides sufficient starting material.

Taking into account the teaching of the prior art (such as, for example, that of WO-A-01/57066 which describes stabilization, solubilization and, related thereto, improved application of hydrophobins due to sulfite treatment; or of WO-A-01/57076 which describes purification of hydrophobin via adsorption to Teflon beads and elution by means of a detergent such as Tween at low temperatures; or of WO-A-01/57528 which describes fixing hydrophobins to surfaces by applying Tween and temperatures up to 85 degrees Celsius), a very large variety of solid materials such as glass, fibers, fabrics, leather, painted objects, films, facades can be coated with hydrophobin.

The following nonlimiting examples describe the invention in more detail with reference to the attached figures in which

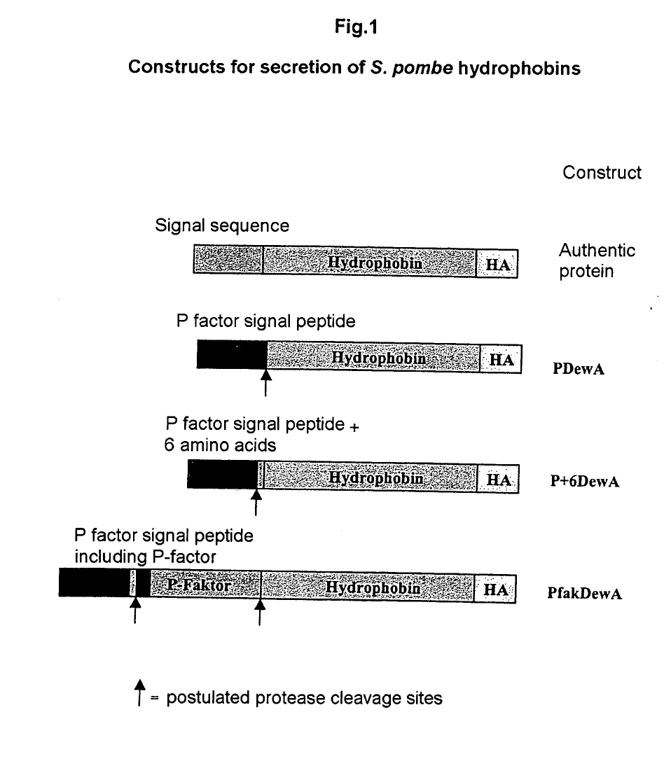

FIG. 1 depicts various constructs, prepared according to the invention, for secretion of hydrophobins from S. pombe.

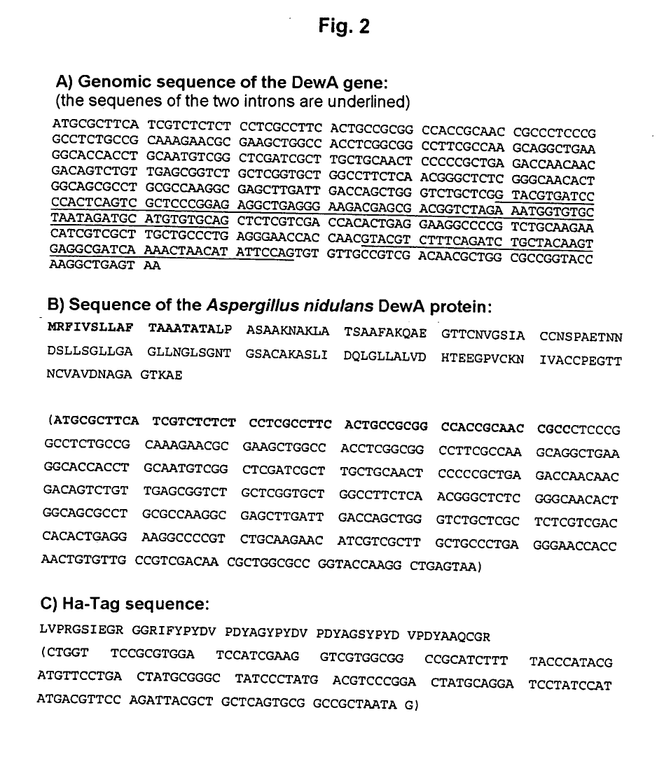

FIG. 2A) depicts the genomic sequence of the DewA gene (SEQ ID NO:39); the sequences of the two introns are underlined; B) depicts the amino acid sequence and, in parentheses, the corresponding DNA sequence of the Aspergillus nidulans DewA protein; the signal sequence is printed in bold type, and the partial sequence following the signal sequence corresponds to the sequence of mature DewA; C) depicts the amino acid sequence and, in parentheses, the corresponding DNA sequence of the HA-Tag.

FIG. 3A) depicts the amino acid sequence and, in parentheses, the corresponding DNA sequence of the P-factor pre-protein; the signal sequence is printed in bold type; the underlined partial sequences following the signal sequence correspond to the sequences of the four mature pheromone peptides; the pheromone next to the signal peptide is referred to as P-factor; B) depicts the amino acid sequence and, in parentheses, the corresponding DNA sequence of the removable signal peptide and, downstream therefrom, the 6 amino acids (underlined) of the P-factor pre-protein; C) depicts the amino acid sequence and, in parentheses, the corresponding DNA sequence of the “P-shuttle” of the invention; the signal sequence is printed in bold type, the underlined partial sequence following the signal sequence corresponds to the sequence of mature P-factor;

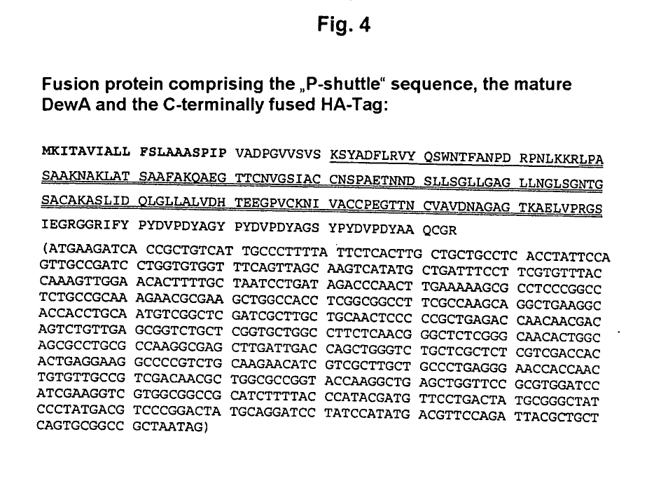

FIG. 4 depicts a fusion protein of the invention, comprising the “P-shuttle” sequence (signal sequence in bold; mature P protein underlined), the mature DewA (double-underlined) and the C-terminally fused HA-Tag (SEQ ID NO:18; encoded by SEQ ID NO:17);

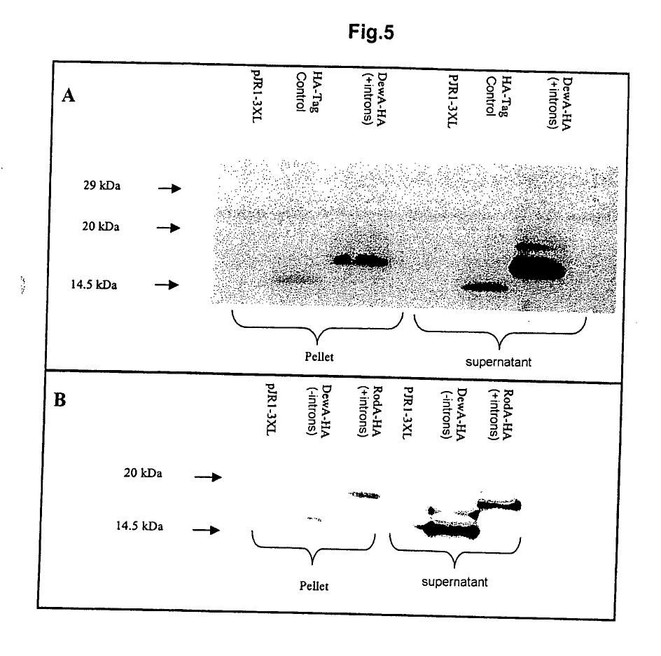

FIG. 5 depicts the immunological detection of hydrophobins in S. pombe* For immunological detection, the A. nidulans hydrophobin genes DewA and RodA were fused to an HA tag, cloned into the pJR1-3XL expression vector and transformed into S. pombe. “Membrane fraction” and “cytosoli proteins” were fractionated by SDS-PAGE. Detection in the Western analysis was carried out using HA antibodies. The size standard in kDa is indicated on the left. A: samples of a culture containing the insert-free vector (pJRI-3XL, negative control), an control) and a vector comprising the HA-tagged DewA gene with introns (DewA-HA(+introns)) were applied. B: samples of a culture containing the vector (pJR1-3XL, negative control), a vector comprising the HA-tagged DewA gene without introns (DewA-HA(−introns)) or the HA-tagged RodA gene with introns (RodA-HA(+introns)) were applied in each case.

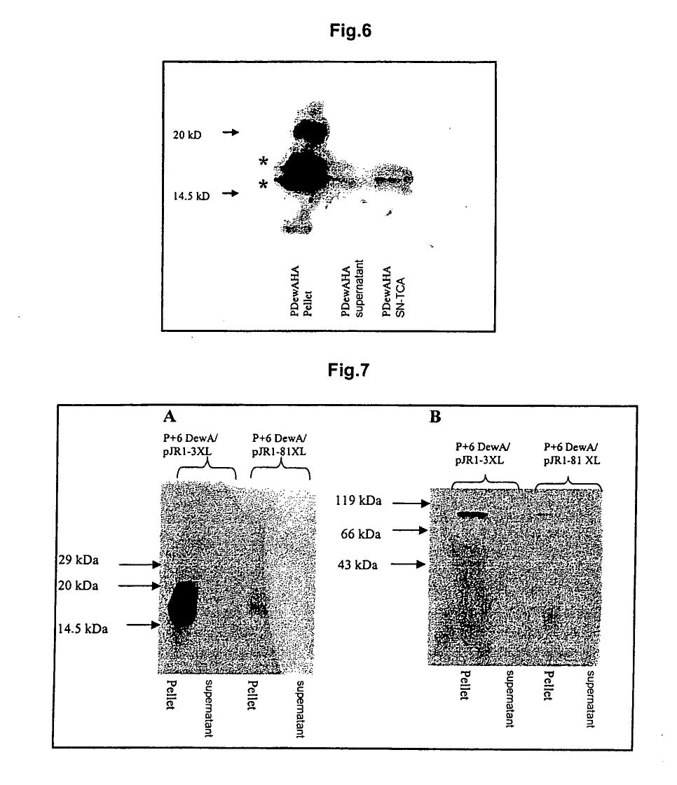

FIG. 6 depicts the immunological detection of the expression of hydrophobins in S. pombe. The PDewAHA protein was expressed in S. pombe. The cells were harvested, the culture supernatant was divided into aliquots and a part was precipitated with TCA. The protein was detected after SDS-PAGE and Western blot with the aid of HA antibodies. The bands indicated by * correspond to the precursor protein (approx. 18 kD, upper band) and the mature form (approx. 17 kD, lower band).

FIG. 7 depicts the detection of expression of hydrophobins in S. pombe. S. pombe cells were transformed with plasmids which express P+6DewA via a strong promoter (pJR1-3XL) or a weaker promoter (pJR1-81XL). The cells carry chromosomally a version of the prp1 gene with a c-myc-Tag which serves as control in order to rule out contamination of the culture supernatant by lysed cells. Cells were harvested (pellet), the culture supernatant was precipitated with TCA (supernatant). The proteins were detected after SDS-PAGE and Western blot with the aid of antibodies against HA (A) or against c-myc (B).

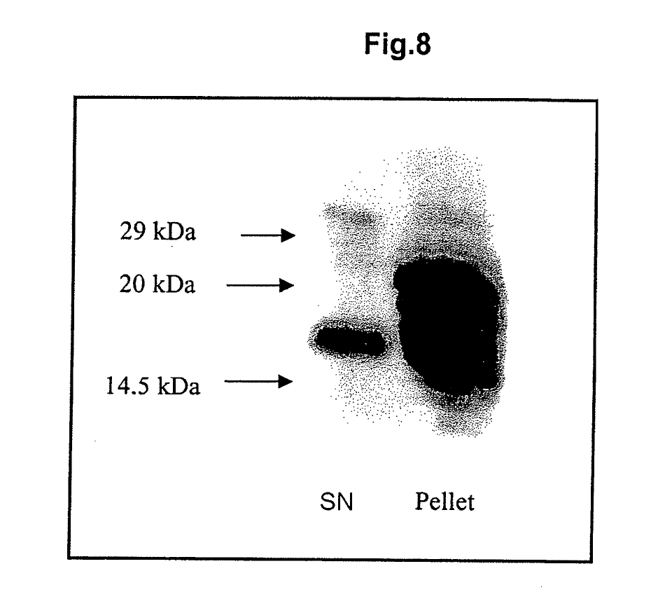

FIG. 8 depicts the detection of secretion with the aid of the “P shuttle” method. S. pombe cells were transformed with plasmids which express PfakDewA via a weaker promoter (pJR1-81XL). The cells were harvested (pellet) and the culture supernatant was precipitated with TCA (SN). The protein was detected after SDS-PAGE and Western blot with the aid of antibodies against HA.

FIG. 9 depicts the three genes which encode in each case the S. pombe M-factor (SEQ ID NO:51 for mature factor): A) depicts sequences for the mfm1⁺⁻ gene; B) depicts sequences for the mfm2⁺⁻ gene; and C) sequences for the mfm3⁺⁻ gene.

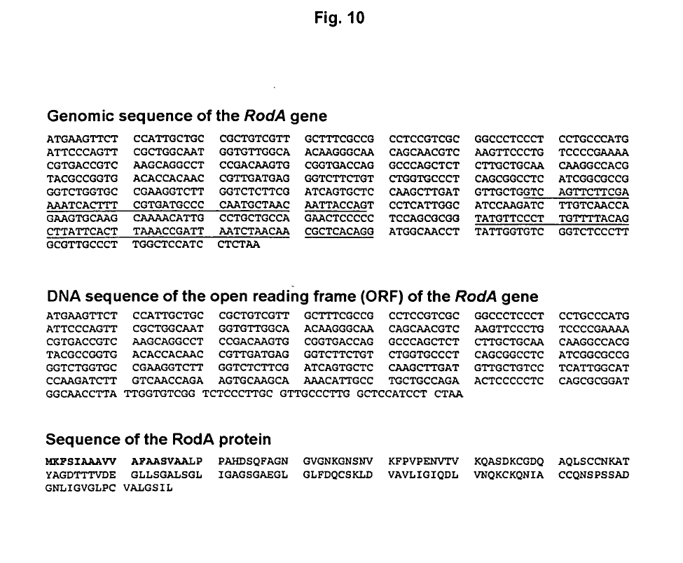

FIG. 10 depicts the RodA gene. The genomic sequence (SEQ ID NO:52) of the RodA gene comprises two introns (underlined) which are not present in the corresponding coding ORF (SEQ ID NO:53). The pre-protein (SEQ ID NO:54) comprises a removable signal sequence (printed in bold type) which is absent in the mature protein (SEQ ID NO:56; encoded by SEQ ID NO:55).

### EXPERIMENTAL SECTION

General Experimental Details:

a) General Cloning Methods

The cloning steps carried out for the purpose of the present invention, such as, for example, restriction cleavages, agarose gel electrophoresis, purification of DNA fragments, transfer of nucleic acids to nitrocellulose and nylon membranes, linking of DNA fragments, transformation of E. coli cells, culturing of bacteria, propagation of phages and sequence analysis of recombinant DNA were carried out as described by Sambrook et al. (1989) loc. cit., unless stated otherwise.

DNA was purified from reaction mixtures or after gel electrophoresis by means of the NucleoSpin Extract Kit (Machery-Nagel, Düren, Germany) and plasmid DNA was isolated from E. coli with the aid of the NucleoSpin Plasmid Quick Pure Kit (Machery-Nagel, Düren, Germany) according to the manufacturer's information.

Restriction enzymes (Invitrogen) were used according to the manufacturer's information. Ligations of DNA were carried out with the aid of T4 ligase (Promega, Mannheim, Germany) according to the manufacturer's information.

Transformations into E. coli were carried out by means of electroporation using the Gene Pulser II apparatus (BIO-RAD, Munich, Germany) and 2-mm electroporation cuvettes (Biozym Diagnostik, Hess. Oldendorf, Germany) according to the manufacturers' information. Transformants were selected on LB medium (Lennox, 1955, Virology, 1:190) containing ampicillin (150 mg/l).

b) Polymerase Chain Reaction (PCR)

PCR amplifications were carried out with the aid of Combizyme DNA polymerase (Invitek, Berlin, Germany) according to the manufacturer's information. In each case 1 pmol of the appropriate primers was used per 100 μl of reaction volume.

c) Culturing

S. pombe was cultured as described in Alfa et al. (Alfa, C., Fantes, P., Hyams, J., McLeod, M. and Warbrick, E. (1993) Experiments with fission yeast. Cold Spring Harbor Laboratory Press, New York) and Gutz et al. (Gutz, H., Heslot, H., Leupold, U. and Loprieno, U. (1974) Schizosaccharomyces pombe. In: Handbook of Genetics 1, pp 395-446, Plenum Press, New York).

Recombinant strains were cultured as described in Alfa et al. (Alfa, C., Fantes, P., Hyams, J., McLeod, M. and Warbrick, E. (1993) Experiments with fission yeast. Cold Spring Harbor Laboratory Press, New York) und Gutz et al. (Gutz, H., Heslot, H., Leupold, U. and Loprieno, U. (1974) Schizosaccharomyces pombe. In: Handbook of Genetics 1, pp 395-446, Plenum Press, New York).

d) Cell Disruption

For quick controls of expression, the cells were removed by centrifugation (5 min, 3.500×g) and the cell pellet was taken up directly in Laemmli buffer (Laemmli, U.K. (1970) Cleavage of structural proteins during the assembly of the head of bacteriophage T4 (Nature 227:680-685)) for gel electrophoresis.

For cell disruption, the cells were harvested by centrifugation at 3.500×g for 5 min. The cell pellets were resuspended in 1 ml of 1×PBS and 1 volume of glass beads was added. The mixture was vortexed for 5 min, and the supernatant above the glass beads was removed.

e) Organisms Used

The strains DH5α (Invitrogen), XL10-Gold (Stratagene) or BL21 (BioLabs) were used for E. coli work.

S. pombe strains were taken from the fission yeast strain collection of the group of Prof. Dr. G. Rödel of the Institute for Genetics of Technische Universität Dresden, Germany.

### Example 1

Preparation of the DewA and DewAHA Expression Constructs and Cloning into Vector pJR1-3XL

a) Isolation of the Genomic DNA Sequence of the DewA Gene and Removal of Introns

A. nidulans chromosomal DNA which had been isolated as in Kaiser et al. (Kaiser, C., Michaelis, S. and Mitchell, A. (1994) Methods in Yeast Genetics. Cold Spring Harbor Laboratory Press, New York) was kindly provided by Prof. Dr. A. Brakhage (Hanover, Germany).

The approx. 550 bp genomic DNA fragment was PCR-amplified using chromosomal DNA as template and the primers SpDewBamrev and ScDewBamfor.

ScDewBamfor:

(SEQ ID NO:41)

5′ - TAA TAA GGA TCC ATG CGC TTC ATC GTC TCT CTC C
- 3′

SpDewBamrev:

(SEQ ID NO:28)

5′ - TAA TAA GGA TCC TTA CTC AGC CTT GGT ACC GGC
-3′

The reaction mixture was fractionated by gel electrophoresis and the corresponding DNA band was eluted as described above. The fragment which is flanked on either side by a BamHl cleavage site introduced by the primers was cleaved with the restriction endonuclease BamHl (Invitrogen) according to the manufacturer's information and purified from the reaction mixture (see above).

The vector pUC18 (Yanisch-Pron, C., Vieira, J. and Messing, J. (1985) Improved M13 phage cloning vectors and host strains: Nucleotide sequences of M13mpl8 and pUC19 vectors. Gene 33:103) was likewise cleaved with BamHl, fractionated by gel electrophoresis and subsequently eluted from the gel (see above).

Vector and fragment were ligated (see above) and the ligation mixture was transformed into E. coli. Recombinant plasmids were identified after plasmid preparation and subsequent restriction digestion. After cloning, the correct DNA sequence of the cloned PCR products was verified by sequencing, and this was also done for all constructs prepared hereinbelow. Sequencing reactions were carried out according to Sanger et al. (Sanger, F., Nicklen, S. and Coulson, A. R. (1977) DNA sequencing with chain terminating inhibitors. Proc Natl Acad Sci USA 74:5463-5467). The sequencing reactions were carried out with the aid of the “Thermo-Sequenase fluorescent labelled primer cycle sequencing kit with 7-deaza-dGTP” (Amersham Pharamacia Biotech, Freiburg, Germany) and 5′ IRD800-labeled primers (MWG Biotech AG, Ebersberg, Germany). The products were fractionated and the sequence was analyzed using the automated sequencing system LI-COR 4000/4200 (MWG Biotech AG, Ebersberg, Germany).

A construct containing the intron-containing, genomic DewA gene cloned into the BamHl cleavage site of the pUC18 vector was referred to as pDewAgen.

The two introns present in the genomic DewA DNA (see genomic DewA sequence SEQ ID NO: 39) were removed by means of “Overlap Extension PCR” (OEP) (cf. Pogulis, R. J., Vallejo, A. N. and Pease, L. R. In vitro recombination and mutagenesis by overlap extension PCR. Methods Mol Biol. 1996; 57:167-76).

Using DNA of the pDewAgen construct as template, subfragments of the open reading frame (ORF) of DewA were first PCR-amplified with the aid of the primer pairs ScDewBamfor/Dewl1rev, Dewl1for/Dewl2rev and Dewl2for/SpDewBamrev.

ScDewBamfor:

(SEQ ID NO:41)

5′ - TAA TAA GGA TCC ATG CGC TTC ATC GTC TCT CTC
C - 3′
Dewl1rev:

(SEQ ID NO:24)

5′ - GT GTG GTC GAC GAG AGC GAG CAG ACC CAG CTG -
3′
Dewl1for:

(SEQ ID NO:23)

5′ - CAG CTG GGT CTG CTC GCT CTC GTC GAC CAC AC -
3′
Dewl2rev:

(SEQ ID NO:26)

5′ - GTC GAC GGC AAC ACA GTT GGT GGT TCC CTC -3′
Dewl2for:

(SEQ ID NO:25)

5′ - GAG GGA ACC ACC AAC TGT GTT GCC GTC GAC -3′
SpDewBamrev:

(SEQ ID NO:28)

5′ - TAA TAA GGA TCC TTA CTC AGC CTT GGT ACC
GGC -3′

These subfragments were fractionated by gel electrophoresis and purified. In the final PCR, the intron-less ORF was amplified with said subfragments as template and the distal primers ScDewBamfor and SpDewBamrev. The approx. 410 bp PCR product was fractionated by gel electrophoresis, purified and cleaved with the restriction endonuclease BamHl. Appropriate cleavage sites have been introduced by the distal primers. The cleaved fragment was purified and cloned into the pUC 18 vector likewise cleaved with BamHl. Vector and fragment were ligated (see above) and the ligation mixture was transformed into E. coli. After plasmid minipreparation, recombinant plasmids were identified and the correct sequence of cloned ORF was verified by sequencing. The construct obtained in this way (pDewAORF) served as template for the construction of corresponding expression plasmids.

b) Preparation of the DewHA(+introns) and DewHA(−introns) Expression Constructs and Introduction of a C-Terminal Hemagglutinin Tag

Since no specific antibodies against DewA are available, DewA was fused by OEP to the HA epitope to detect heterologous expression. First, in the primary PCRs, the primer pairs SpDewXhofor/DewAHArev and DewAHAfor/DewAHANcorev were utilized.

SpDewXhofor:

(SEQ ID NO:27)

5′ - TAA TAA CTC GAG ATG CGC TTC ATC GTC TCT CTC
C -3′
DewAHArev:

(SEQ ID NO:30)

5′ - TCC ACG CGG AAC CAG CTC AGC CTT GGT ACC -3′
DewAHAfor:

(SEQ ID NO:29)

5′ - GGT ACC AAG GCT GAG CTG GTT CCG CGT GGA -3′
DewAHANcorev:

(SEQ ID NO:31)

5′ - ATT ATT CCA TGG CTA TTA GCG GCC GCA CTG AGC
AGC - 3′

DNA of the pDewAgen construct served as template for preparing DewHA(+introns), and DNA of the pDewAORF construct served as template for preparing DewHA(−introns). The vector yEP351HA (Kettner, K., Friederichs, S., Schlapp, T. and Rödel G (2001) Expression of a VEGF-like protein from Parapoxvirus ovis in the yeasts Saccharomyces cerevisiae and Schizosaccharomyces pombe. Protein Expr Purif August 22(3):479-83) which carries the HA-tag DNA sequence was utilized as template as template for PCR with DewAHAfor/DewAHANcorev. The DNA coding for the HA epitope was fused to the particular DewA-DNA in the final PCRs by means of the primer pair SpDewXhofor/ DewAHANcorev and the respective subfragments. The fragments amplified in this way are flanked 5′ by an XhoI restriction cleavage site and 3′ by an NcoI restriction cleavage site, both of which were introduced with the aid of the distal primer. The fragments were fractionated by gel electrophoresis, purified and cleaved with the restriction endonucleases XhoI and NcoI and purified from the reaction mixture.

The vector pJR1-3XL (Moreno, M. B., Duran, A. and Ribas, J. C. A family of multifunctional thiamine-repressible expression vectors for fission yeast. Yeast. 2000 Jun. 30; 16(9):861-72) was likewise cleaved with the restriction enzymes XhoI and NcoI, fractionated by gel electrophoresis and purified. Vector and fragment were ligated (see above) and the ligation mixture was transformed by electroporation into E coli. After plasmid minipreparation, recombinant plasmids were identified and the correct sequence of the cloned ORF was verified by sequencing. In the DewA-HA(+introns) and DewA-HA(−introns) expression plasmids obtained in this way, expression of the fusion constructs in S. pombe is under the control of the strong nmt1 promoter.

c) Expression of DewA-HA(+intron) and DewA-HA(−intron)

The DewA-HA(+introns) and DewA-HA(−introns) vectors obtained according to a) and b) were transformed into the S. pombe host strain KO103 (h^{-s} ade6-M210 leu1-32 his7-366), as described by Schiestl and Gietz (Schiestl, R. H. and Gietz, R. D. (1989) High efficiency transformation of intact yeast cells using single stranded nucleic acids as a carrier. Curr Genet 16:339-346). The leucine auxotrophy of the S. pombe strain, caused by the leu1-32 mutation, is complemented by the S. cervisiae LEU2 gene present on the expression vectors. It is thus possible to select transformants on minimal medium without leucine. Expression of the fusion proteins in corresponding yeast transformants was studied by means of Western blot analyses.

The antibodies Anti-HA (article 1 583 816, anti-HA (12CA5) mouse monoclonal antibody) and anti-c-myc (article 1 667 149, anti-c-myc antibody) were purchased from Roche Diagnostics (Mannheim, Germany).

After culturing, yeast transformants were harvested, disrupted with glass beads, and the 3.500×g centrifugation supernatant was removed. In each case 50 μg of the 20.000×g pellets (“membrane fraction”) and of the supernatant (“cytosolic proteins”) were applied and fractionated in an SDS-PAGE. Detection in the Western analysis was carried out using HA antibodies. FIG. 5 depicts the result. The size standard in kDa is indicated on the left. In FIG. 5A, samples of a culture containing the insert-free vector (PJR1-3XL, negative control), an HA-tagged control protein (positive control) or a vector comprising the HA-tagged DewA gene with introns (DewA-HA(+introns)) were applied. In FIG. 5B, samples of a culture containing the vector (pJR1-3XL, negative control), or a vector comprising the HA-tagged DewA gene without introns (DewA-HA(−Introns)) or the HA-tagged RodA gene with introns (Rod-AHA(+introns)) were applied in each case.

RodAHA(+introns) was prepared analogously to the information of Example 1 a) and 1 b). RodA is another hydrophobin from A. nidulans.

### Example 2

Preparation of expression Vectors for Secretion of the Expressed DewA Vector Comprising the PDewAHA Construct

a) Preparation of the PDewAHA Construct Comprising the Coding Sequence for P-Factor Signal Peptide

In order to optimize secretion of the protein from S. pombe cells, the authentic secretion signal of the A. nidulans protein, which is not effective in fission yeast, was first replaced by the removable signal peptide of the S. pombe P factor. The P factor is secreted as peptide pheromone into the medium by the cells. It is synthesized in the cell as precursor protein (pre-protein) consisting of a removable N-terminal signal sequence and four copies of P factor, in each case separated by short spacer sequences, and, in the course of secretion, undergoes maturation, including removal of the signal sequence and proteolytic release of the four P-factor Peptides.

The P-factor signal sequence was first amplified by means of PCR and S. pombe genomic DNA as template, using the primer pair SigPXhofor/PDewArev, and the corresponding PCR product was purified.

SigPXhofor:

(SEQ ID NO:34)

5′ - TAA TTT CTC GAG ATG AAG ATC ACC GCT GTC ATT
GCC CTT TTA TTC TCA C -3′
PDewArev:

(SEQ ID NO:33)

5′ - GGC AGA GGC CGG GAG TGG AAT AGG TGA GGC -3′

The PCR product of the primer pair PDewfor/DewAHANcorev and DNA of the DewA-HA(−introns) construct as template was likewise fractionated by gel electrophoresis and purified.

PDewfor:

(SEQ ID NO:32)

5′ - GCC TCA CCT ATT CCA CTC CCG GCC TCT GCC -3′
DewAHANcorev:

(SEQ ID NO:31)

5′ - ATT ATT CCA TGG CTA TTA GCG GCC GCA CTG AGC
AGC -3′

These two primary PCR products were used as templates in the final PCR with the distal primers SigPXhofor/DewAHANcorev. The PDewAHA fragment amplified in this way is flanked 5′ by an XhoI restriction cleavage site and on its 3′ end by an NcoI restriction cleavage site, both of which were introduced with the aid of the above primers. In the fusion protein encoded by this fragment (PDewAHA), the removable signal sequence of A. nidulans DewA has been replaced by the removable signal peptide of the P-factor precursor protein.

The PDewAHA fragment was cleaved with the restriction endonucleases XhoI and NcoI, fractionated by gel electrophoresis and ligated into the pJR1-3XL vector (see above), which had been cleaved with the same restriction endonucleases. Vector and fragment were ligated (see above) and the ligation mixture was transformed by electroporation into E. coli. After plasmid minipreparation, recombinant plasmids were identified and the correct sequence of the cloned ORF was verified by sequencing. The construct obtained was referred to as PDewAHA.

b) Expression

The experiments were carried out analogously to Example 1c).

The PDewAHA protein was expressed in S. pombe. The cells were harvested, the culture supernatant was divided into aliquots and a part was precipitated with TCA. The TCA precipitate was taken up in Laemmli buffer (Laemmli, U.K. (1970) Cleavage of structural proteins during the assembly of the head of bacteriophage T4 (Nature 227:680-685)). Cell pellets, supernatant and TCA-precipitated supernatant were studied. The protein was detected after SDS-PAGE and Western blot with the aid of HA antibodies. FIG. 6 depicts the result. The bands indicated by * correspond to the precursor protein (approx. 18 kD, top band) and the mature form (approx. 17 kD, bottom band).

The analysis revealed that no effective secretion was noticeable. The N-terminal fusion of the removable P-factor signal peptide is not sufficient for secretion of the fused hydrophobin.

### Example 3

Preparation of Expression Vectors for Secretion of the Expressed DewA Vector Comprising the P+6DewAHA Construct

a) Preparation of the P+6DewAHA Construct Comprising the Coding Sequence for P-factor Signal Peptide, C-Terminally Extended by 6 Amino Acids

In order to ensure an authentic sequence environment of the signal peptide, which may be important for secretion, the sequence of said signal peptide in the fusion protein was extended by the 6 C-terminally adjoining amino acids (P+6DewA) by means of OEP using the primer pair SigPXhofor/P+6DewArev and P+6DewAfor/DewAHANcorev and DNA of the PDewAHA construct as template in the primary PCR reactions

SigPXhofor:

(SEQ ID NO:34)

5′ - TAA TTT CTC GAG ATG AAG ATC ACC GCT GTC ATT
GCC CTT TTA TTC TCA C -3′
P + 6DewArev:

(SEQ ID NO:36)

5′ - CAC ACC AGG ATC GGC AAC TGG AAT AGG TGA
GGC -3′
P + 6DewAfor:

(SEQ ID NO:35)

5′ - GTT GCC GAT CCT GGT GTG CTC CCG GCC TCT
GCC -3′
DewAHANcorev:

(SEQ ID NO:31)

5′ - ATT ATT CCA TGG CTA TTA GCG GCC GCA CTG AGC
AGC -3′

and the primer pair SigPXhofor/DewAHANcorev in the final PCR reaction.

The P+6DewA fragment was cleaved with the restriction endonucleases XhoI and NcoI, fractionated by gel electrophoresis and ligated into the pJR1-3XL vector (see above) which had been cleaved with the same restriction endonucleases, and the ligation mixture was transformed by electroporation in E. coli. After plasmid minipreparation, recombinant plasmids were identified and the correct sequence of the cloned ORF was verified by sequencing. The P+6DewA fragment was also cloned into the pJR-81XL vector. Here, transcription of the fusion gene is under the control of the weak nmt81 promoter. This construct was intended to be used for testing a negative influence of the very high transcription in pJR1-3XL constructs on secretion.

The corresponding constructs were referred to as P+6DewA/pJR1-3XL and P+6DewA/pJR1-81XL.

The experiment was carried out analogously to Example 2a.

Cloning of the amplified sequences in pJR1-3XI was carried out analogously to Example 2a.

b) Expression

Expression was carried out analogously to Example 2b).

S. pombe cells were transformed with the two plasmids which express P+6DewA via a strong promoter (pJR1-3XL) and a weaker promoter (pJR1-81XL). The cells carry chromosomally a version of the prp1 gene with a c-myc Tag which serves as a control in order to rule out contamination of the culture supernatant by lysed cells. The cells were harvested (pellet), and the culture supernatant was precipitated with TCA (supernatant). The precipitate was taken up in Laemmli buffer and likewise analyzed. The proteins were detected after SDS-PAGE and Western blot with the aid of antibodies against HA (FIG. 7A) and against c-myc (Roche Diagnostics) (FIG. 7B).

As FIG. 7 illustrates, this construction is also unsuitable for effective secretion.

### Example 4

Preparation of Expression Vectors for Secretion of the Expressed DewA Vector Comprising the PfakDewAHA Construct

a) Preparation of the PfakDewAHA Construct Comprising the Sequence Coding for the Mature First P Factor, Including the P-Factor Signal Peptide

DewAHA was fused by means of OEP to the carboxyl-terminal end of the sequence of the mature P factor. The PCR fragments obtained in the primary PCR reactions using the primer pairs SigPXhofor/PfakDewArev and S. pombe genomic DNA as template

SigPXhofor:

(SEQ ID NO:34)

5′ - TAA TTT CTC GAG ATG AAG ATC ACC GCT GTC ATT
GCC CTT TTA TTC TCA C -3′
PfakDewArev:

(SEQ ID NO:38)

5′ - GGC AGA GGC CGG GAG GCG CTT TTT CAA GTT GGG
TC -3′
and PfakDewAfor/DewAHANcorev and DNA of the
P + 6DewA/pJR1-81XL construct as template
PfakDewAfor:

(SEQ ID NO:37)

5′ - AAC TTG AAA AAG CGC CTC CCG GCC TCT GCC -3′
DewAHANcorev:

(SEQ ID NO:31)

5′ - ATT ATT CCA TGG CTA TTA GCG GCC GCA CTG AGC
AGC -3′

were separated by gel electrophoresis, purified and utilized as template for the final PCR with the aid of the primer pair SigPXhofor/ DewAHANcorev. The PfakDewA fragment obtained in this way was cleaved with the restriction endonucleases XhoI and NcoI, fractionated by gel electrophoresis and ligated into the pJR1-81XL vector (see above) which had been cleaved with the same restriction endonucleases. The ligation mixture was transformed by electroporation into E coli. After plasmid minipreparation, recombinant plasmids were identified and the correct sequence of the cloned ORF was verified by sequencing. Such a construct was referred to as PfakDewA/pJR1-81XL. In this construct, the fused sequence coding for the P-factor pre-protein including the first amino-terminal pheromone and for the hydrophobin is under the control of the nmt81 promoter.

The experiment was carried out analogously to Example 2a, but with the pJR1-81XL expression vector being used.

The amplified sequences were cloned into pJR1-81XL analogously to Example 2a. For this purpose, the amplified DNA which had been cleaved with the restriction endonucleases XhoI and NcoI was cloned into the XhoI and NcoI cleavage sites of the pJR1-81XL expression vector.

b) Expression

Expression was carried out analogously to Example 3b).

The cells were pelleted and the culture supernatant was precipitated with TCA. The precipitate was taken up in Laemmli buffer and likewise analyzed. The protein was detected after SDS-PAGE and Western blot with the aid of antibodies against HA. FIG. 8 depicts the result which shows that effective secretion of the hydrophobin into the medium is achieved with the aid of this construction. Thus, the corresponding fusion protein comprises all P-factor pre-protein sequences required for secretion in their authentic context. The P factor itself is proteolytically removed.

### Example 5

Microscopic Detection of Adsorption of Expressed Hydrophobin to Teflon

The microscopic detection of adsorption of expressed hydrophobin to Teflon makes use of a fluorescently labeled HA antibody (Molecular Probes, Cat. No. A-21287).

Transformed host cells prepared according to any of Examples 1 to 4 are cultured. Cells and, where appropriate, supernatant are harvested separately. Cells which have been transformed with a corresponding vector without hydrophobin gene and cultured and corresponding culture supernatants serve as reference samples.

The cells are mechanically disrupted (vibratory mill). Teflon platelets are incubated in the cell disruption solution or in the supernatant at room temperature for 18 h, and rinsed with water (3×10 min). The treated Teflon is then incubated in PBS with fluorescently labeled antibody, followed by rinsing again with PBS (3×15 min) and drying in an N₂jet. Finally, the evaluation is carried out in a fluorescence microscope.

No fluorescence is observed on the reference sample (results not shown), but spot-like fluorescence is obvious after incubation in the cell homogenate or culture supernatant (when hydrophobin is secreted by the expressing cells).

1. An expression construct, which comprises a nucleic acid sequence coding for a shuttle peptide construct which is processable by yeast cells, has the formula
(Sig-SP),
and comprises in 5′-3′ orientation the nucleic acid sequences coding for
a) a signal peptide (Sig) processably linked to
b) at least one shuttle peptide (SP) secretable by said yeast cells.

2. The expression construct as claimed in claim 1, wherein the shuttle peptide construct (Sig-SP) is derived from polypeptide processed by yeasts of the genus Schizosaccharomyces.

3. The expression construct as claimed in claim 1, wherein the shuttle peptide construct (Sig-SP) is derived from a pheromone pre-protein of a yeast, said pheromone (Pher) being derivable from the pre-protein and secretable by N- and C-terminal processing.

4. The expression construct as claimed in claim 3, wherein the signal polypeptide (Sig) is the proteolytically removable native signal polypeptide of the pheromone pre-protein.

5. The expression construct as claimed in claim 4, wherein the C-terminally processed pheromone (Pher) comprises a C-terminal protease cleavage site.

6. The expression construct as claimed in claim 1, further comprising a nucleic acid sequence coding for a homologous or heterologous target protein (Targ) processably linked to the C-terminus of the shuttle peptide construct (Sig-SP).

7. The expression construct as claimed in claim 1 comprising a nucleic acid sequence coding for a fusion protein which is processable by yeast cells and has the formula
Sig-L1ₙ-Pher-L2ₘ-Targ
in which
Sig is signal polypeptide, Pher is processed pheromone, and Targ is target protein,
L1 and L2 are processable linkers and
n and m are independently of one another, and is 0 or 1.

8. The expression construct as claimed in claim 1, wherein the nucleic acid sequence coding for the shuttle peptide construct (Sig-SP) comprises a signal polypeptide-coding sequence according to SEQ ID NO: 3 or a functional equivalent thereof which is operatively linked to the nucleic acid sequence according to SEQ ID NO: 5, coding for a mature pheromone protein (P factor), or to a functional equivalent thereof.

9. The expression construct as claimed in claim 1, wherein the nucleic acid sequence coding for the shuttle peptide construct comprises a sequence according to SEQ ID NO: 1, optionally extended at the 3′ end by the sequence coding for a target protein (Targ).

10. The expression construct as claimed in claim 6, wherein the target protein is a hydrophobin, in particular a class I hydrophobin.

11. The expression construct as claimed in claim 10, wherein the hydrophobin is selected from the group consisting of SEQ ID NO: 14 (DewA), SEQ ID NO: 19 (RdlA), SEQ ID NO: 20 (RdlB), SEQ ID NO: 21 (HYP1) and SEQ ID NO: 22 (HYP4), or is encoded by a nucleic acid sequence according to SEQ ID NO: 13.

12. An expression vector comprising the expression construct as claimed in claim 1 which is operatively linked to at least one regulatory nucleic acid sequence.

13. A recombinant microorganism, comprising at least one expression construct as claimed in claim 1 stably integrated into the host genome.

14. The microorganism as claimed in claim 13, selected from among yeasts.

15. The microorganism as claimed in claim 14, selected from among yeasts of the genus Schizosaccharomyces.

16. A shuttle peptide construct, processable by yeast cells and derived from a pheromone pre-protein of a yeast, wherein the pheromone is derivable from said pre-protein and secretable by N- and C-terminal processing.

17. The shuttle peptide construct as claimed in claim 16, comprising a signal polypeptide N-terminally processably linked to the C-terminally processed pheromone polypeptide.

18. The shuttle peptide construct as claimed in claim 17, wherein the signal polypeptide is the proteolytically removable native signal polypeptide of the pheromone pre-protein.

19. The shuttle peptide construct as claimed in claim 17, wherein the C-terminally processed pheromone polypeptide comprises the C-terminal protease cleavage site.

20. The shuttle peptide construct as claimed in claim 16 comprising an amino acid sequence as defined in SEQ ID NO: 2 or a functional equivalent thereof.

21. A method for recombinant preparation of a target protein, which comprises culturing the microorganism as claimed in claim 13, expressing the nucleic acid sequence encoding said target protein and isolating the target protein secreted into the culture medium.

22. The method as claimed in claim 21, wherein the target protein is a hydrophobin.

23. A nucleic acid, coding for the shuttle peptide construct as claimed in claim 16.

24. A nucleic acid coding for the expression construct as claimed in claim 1.

25. A hydrophobin obtained by the method as claimed in claim 22.

26-28. (canceled)

29. A method of treating the surface of an object comprising obtaining a hydrophobin by the method according to claim 22, and treating the surface of the object with the hydrophobin.

30. The method according to claim 29, wherein the object is selected from the group consisting of glass, fibers, fabrics, leather, painted objects, films and facades.

31. A method of treating the surface of fibers, fabrics and leather, wherein the method comprises obtaining a class I hydrophobin or a hydrophobin as defined in claim 11, and treating the surface of the fibers, fabrics and leather with the hydrophobin.

32. The expression construct of claim 2, wherein the yeast is S. pombe.

33. The microorganism of claim 15, wherein the yeast is S. pombe.
